(19) United States
(12) Patent Application Publication
(10) Pub. No.: US 2010/0260673 A1
(43) Pub. Date: Oct. 14, 2010

(54) PHAGE DISPLAY PEPTIDE PROBES FOR IMAGING EARLY RESPONSES TO ANTIANGIOGENIC TREATMENT

(76) Inventors: QIZHEN CAO, MOUNTAIN VIEW, CA (US); XIAOYUAN CHEN, UNION CITY, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/755,009

(22) Filed: Apr. 6, 2010

Related U.S. Application Data
(60) Provisional application No. 61/212,391, filed on Apr. 10, 2009.

(51) Int. Cl. A61K 51/00; C12Q 1/70; C12N 7/00; C07K 7/08; A61K 38/10; A61K 49/00; A61P 35/00; A61B 5/055

(52) U.S. Cl. 424/1.69; 435/5; 435/235.1; 530/327; 514/14; 424/9.1; 424/9.6; 424/9.3

(57) ABSTRACT

Rapid assessment of cancer response to a therapeutic regimen can determine efficacy early in the course of treatment. Briefly described, embodiments of this disclosure, among others, encompass a class of molecular imaging probes that can predict tumor early responses to anti-angiogenic therapies, such as that based on Bevacizumab (AVASTIN™). In particular, the present disclosure provides peptides that selectively bind to vascularized taget tissues such as, but not limited to solid tumors, responsive to anti-angiogenic therapies and which can, therefore, be useful to selectively concentrate moieties such as detectable labels, or therapeutic agents, in a tumor. The detectable labels, therefore, provide a way to selectively detect and monitor tissues, and most advantageously tumors, that respond to anti-angiogenic therapies.

### CROSS-REFERENCE TO RELATED APPLICATION

This application claims priority to the following U.S. provisional application: “PHAGE DISPLAY PEPTIDE PROBES FOR IMAGING EARLY RESPONSES TO ANTIANGIOGENIC TREATMENT,” having Ser. No. 61/212,391, filed on Apr. 10, 2009, which is entirely incorporated herein by reference.

### STATEMENT REGARDING FEDERALLY SPONSORED RESEARCH OR DEVELOPMENT

This invention was made with government support under Grant Nos.: (P50 CA114747, U54 CA119367, and R24 CA93862), each were awarded by the National Cancer Institute (NCI) and the Intramural Research Program, NIBIB, NIH. The government has certain rights in the invention.

### TECHNICAL FIELD

The present disclosure is generally related to peptides for specifically monitoring tumors responsive to anti-angiogenic therapies, and to methods of using such peptides as imaging agents.

### BACKGROUND

Tumors implanted into isolated perfused organs fail to grow beyond a few millimeters in diameter without angiogenesis (Browder et al., (2000) Cancer Res. 60: 1878-1886; Holash et al., (1999) Science 284: 1994-1998). Consequently, antiangiogenic and anti-vascular agents have been intensively investigated for tumor therapy. By targeting tumor vasculature, anti-angiogenic agents do not need to overcome the physiological barriers within tumors (Jain R. K. (1998) Nat. Med. 4: 655-657). In addition, local and circulating endothelial cells are considered genetically stable, so they will presumably resist changes by genetic and epigenetic mechanisms (Browder et al., (2000) Cancer Res. 60: 1878-1886).

Bevacizumab™ (alternatively named AVASTIN™), a humanized monoclonal antibody directed against human vascular endothelial growth factor (VEGF), was the first antibody drug developed as an inhibitor of angiogenesis to be approved by the Food and Drug Administration (FDA) (Ferrara N. (2004) Endocrinol. Rev. 25: 581-611; Hurwitz et al., (2004) New Engl. J. Med. 350: 2335-2342; Kerbel R. S. (2006) Science 312: 1171-1175). Bevacizumab (AVASTIN™) neutralizes all isoforms of human VEGF and inhibits VEGF-induced proliferation of endothelial cells. A combination of Bevacizumab (AVASTIN™) with paclitaxel resulted in marked suppression of tumor growth in both the CWR22R androgen-independent xenograft model of prostate cancer and in the OVCAR3 ovarian tumor model (Fox et al., (2002) Clin. Cancer Res. 8: 3226-3231; Hu et al., (2002) Am. J. Pathol. 161: 1917-24). It has also been reported that Bevacizumab (AVASTIN™) could reverse the protective effect on endothelial cells of the high levels of VEGF produced by the tumor (Sweeney et al., (2001) Cancer Res. 61: 3369-3372).

The conventional standard to evaluate therapeutic response is tumor volume change. Clinical trials with cytotoxic chemotherapeutic agents have mainly used morphological imaging, and in particular, computed tomography (CT) and magnetic resonance imaging (MRI), according to the Response Evaluation Criteria in Solid Tumors (RECIST) introduced in the year 2000 (Jaffe C. C. (2006) J. Clin. Oncol. 24: 3245-3251) to provide indices of therapeutic response. However, anti-angiogenic agents are typically cytostatic rather than cytotoxic, leading to a stop or delay in tumor progression, rather than tumor shrinkage. Thus, tumor volume is an insensitive indicator for evaluation of therapeutic efficacy, and moreover may take months or years to assess. Currently, microvessel density (MVD) is the most commonly used end-point for assessing anti-angiogenic treatment in clinical studies. MVD is measured from biopsies taken before and at one or more times after treatment is complete, using a variety of immunohistochemical vascular markers to identify the vessels (Willett et al., (2004) Nat. Med. 10: 145-147).

However, measurement of MVD is problematic for assessing the vascular efficacy of anti-angiogenic agents (Fujio & Walsh (1999) J. Biol. Chem. 274:16349-16354), since blocking of angiogenesis may be accompanied by a proportional reduction in tumor growth that would not result in a net change in MVD. Besides, vessel counts and/or density measurements may remain unchanged even in the event of effective therapy (Hlatky et al., (2002) J. Natl. Cancer Inst. 94: 883-893). A similar problem has also been found with non-invasive imaging methods for measuring functional vascular volume, such as positron emission tomography (PET) studies with ¹⁵O-oxygen (Miller et al., (2005) Breast Cancer Res. Treat. 89: 187-197), contrast enhanced ultrasound (CEU) (Hughes et al., (2006) IEEE Trans. Ultrason Ferroelectr. Freq. Control 53: 1609-1616), and dynamic contrast-enhanced MRI (DCE-MRI) (Padhani A. R. (2003) Br. J. Radiol. 76: Spec No 1:S60-80), that is, absence of an effect on vascular volume by non-invasive imaging cannot be interpreted as absence of anti-angiogenic effect (Tozer G. M. (2003) Br. J. Radiol. 76: Spec No 1:S23-35).

Biomarkers have great value in early efficacy and safety evaluations, disease diagnosis/staging, indicating disease prognosis, and prediction/monitoring of clinical response to a given intervention. Recently, molecular imaging with biological markers has emerged to provide valuable information at the structural/functional and/or molecular level. Compared with relatively large biomolecules, such as antibodies and proteins, small peptides have advantages as potential probes for molecular imaging. The display of peptide libraries on the surface of bacteriophage (phage) offers a way of searching for peptides with specific binding properties. Phage display peptide libraries are commonly used to obtain defined peptide sequences that interact with a particular molecule. The strength of this technology is its ability to identify interactive regions of proteins and other molecules without preexisting notions about the nature of the interaction. Especially, in vivo phage display selection procedures offer an advantage over in vitro screening protocols in that phages can be selected based on desired pharmacokinetic properties, including delivery and tumoral accumulation.

Recently, in vivo phage display has been explored as a means to identify phage and corresponding peptides with optimal tumor targeting properties in the context of living animals (Han et al., (2008) Nat. Med. 14: 343-349). Moreover, many of these peptides bind to endothelial cell markers, but not directly to tumor cells (Arap et al., (1998) Science 279: 377-380; Pasqualini & Ruoslahti (1996) Nature 380: 364-366).

### SUMMARY

Rapid assessment of cancer response to a therapeutic regimen can determine efficacy early in the course of treatment. Briefly described, embodiments of this disclosure, among others, encompass a class of molecular imaging probes that can predict tumor early responses to anti-angiogenic therapies, such as that based on Bevacizumab (AVASTIN™). In particular, the present disclosure provides peptides that selectively bind to vascularized taget tissues such as, but not limited to solid tumors, responsive to anti-angiogenic therapies and which can, therefore, be useful to selectively concentrate moieties such as detectable labels, or therapeutic agents, in a tumor. The detectable labels, therefore, provide a way to selectively detect and monitor tissues, and most advantageously tumors, that respond to anti-angiogenic therapies.

One aspect of the present disclosure, therefore, provides embodiments of a method for identifying an anti-angiogenic therapy-responsive peptide, comprising: (a) providing a subject animal or human having a target tissue responsive to an anti-angiogenic therapeutic agent; (b) administering an anti-angiogenic therapeutic agent to the subject animal or human; (c) delivering to the subject animal or human a phage-displayed peptide library under conditions allowing at least one bacteriophage species from the phage-displayed peptide library to selectively bind to an anti-angiogenic therapy-responsive site of the target tissue; (d) isolating the target tissue from the subject animal or human; (e) isolating from the tumor a population of bacteriophages; (f) amplifying said population of bacteriophages in a bacterial host and harvesting said amplified bacteriophages; (g) repeating steps (b)-(f), thereby enriching the isolated phage population for peptide-bearing phage comprising an anti-angiogenic therapy-responsive peptide; and (h) determining the nucleotide sequence encoding a phage-displayed peptide isolated by steps (a)-(g), thereby identifying a peptide positively responsive to an anti-angiogenic therapy.

In embodiments of this aspect of the disclosure, the tissue can be a pathological tissue. In some embodiments of this aspect of the disclosure, the pathological tissue is a tumor.

In one embodiment of this aspect of the disclosure, the anti-angiogenic therapeutic agent may be, but is not limited to, bevacizmuab (AVASTIN™).

Another aspect of the present disclosure encompasses isolated bacteriophages comprising an anti-angiogenic therapy-responsive peptide, wherein the isolated bacteriophage is characterized as selectively binding to a vascularized target tissue responsive to an anti-angiogenic therapeutic agent, or cells isolated therefrom.

In some embodiments of this aspect of the disclosure, the anti-angiogenic therapy-responsive peptide may comprise an amino acid sequence selected from the group consisting of: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, where said peptide is characterized as having selective affinity for a vascularized target tissue responsive to an anti-angiogenic therapeutic agent, or cells isolated therefrom.

In embodiments of this aspect of the disclosure, the isolated bacteriophage can further comprise a moiety or plurality of moieties attached to the bacteriophage and selected from the group consisting of: a therapeutic agent, a detectable label, and a combination thereof. In embodiments of this aspect of the disclosure, the detectable label can be selected from the group consisting of: a fluorescent label, a PET detectable label, an MRI-detectable label, and a radioactive label.

Yet another aspect of the disclosure provides isolated anti-angiogenic therapy-responsive peptides that can comprise an amino acid sequence selected from the group consisting of: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, where the peptide is characterized as having selective affinity for a site of a vascularized target tissue responsive to an anti-angiogenic therapeutic agent, or cells isolated therefrom.

Still another aspect of the disclosure provides compositions selective for an anti-angiogenic therapy-responsive tissue in a subject animal or human comprising an anti-angiogenic therapy-responsive peptide linked to a moiety or plurality of moieties desired to be delivered to a tissue characterized as selectively binding anti-angiogenic therapy-responsive peptide, where the peptide is characterized as having selective affinity for a vascularized tissue responsive to an anti-angiogenic therapeutic agent, or cells isolated therefrom.

Yet another aspect of the disclosure provides embodiments of methods of imaging a tissue in an animal or human subject, comprising: (a) delivering to a subject animal or human a pharmaceutically acceptable composition comprising an anti-angiogenic therapy-responsive peptide linked to a detectable label, where the anti-angiogenic therapy-responsive peptide is characterized as selectively concentrating at a site in a vascularized target tissue; and (b) detecting the label in the subject animal or human, thereby identifying the location of an anti-angiogenic therapy-responsive vascularized target tissue in the host.

In some embodiments of this aspect of the disclosure, the method can further comprise the steps of: (i) delivering to the subject a therapeutic agent; (ii) periodically imaging the vascularized target tissue in the subject; and (iii) determining from the image size of the tissue whether the therapeutic agent is effective in reducing the size of the tumor in the subject.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates a method of identifying peptides selectively concentrating in tumors that are responsive to anti-angiogenic therapy.

FIG. 2 illustrates the amino acid sequences of six peptides (SEQ ID NOs.: 1-6) selectively responsive in tumors responsive to anti-angiogenic therapy.

FIG. 3 illustrates a graph showing that the xenograft LS174T colorectal cancer is responsive to Bevacizumab (AVASTIN™) therapy.

FIG. 4A is a digital near-infrared fluorescence image at 24 h after intravenous injection of IRdye800-labeled LLADTTHHRPWT-bearing phages (1 nmol dye per mouse) showing prominent tumor phage particle accumulation in Bevacizumab (AVASTIN™)-treated, but not saline control mice LS174T tumors. Mice were imaged 1 day after the three doses of Bevacizumab (AVASTIN™) (the fifth day following the start of treatment), at which time no difference in tumor volume between the treatment and control groups was found.

FIG. 4B shows a graph illustrating tumor-to-normal tissue (T/N) ratios of the 2D optical images at 1, 2, 4, 20, and 24 h after administration of optically-labeled phage particles.

FIG. 5 shows digital images illustrating the selective concentration of cy5.5-labeled LLADTTHHRPWT-peptide phages in the LS174T colorectal tumor model responsive to the anti-angiogenesis agent Bevacizumab (AVASTIN™).

FIG. 6 shows a series of digital photomicrographs illustrating the histological detection of phage particles in tumor tissues with Cy5.5-labeled LLADTTHHRPWT-peptide phages and showing that Bevacizumab (AVASTIN™)-treated tumor tissues had more phage homing than did saline control tumor tissues.

FIG. 7 shows a graph illustrating the study of cellular toxicity of human umbilical vein endothelial cells (HUVEC), LS174T human colorectal cancer cells, and 4T1 murine breast cancer cells were subjected to a colorimetric MTT assay after adding AVP phage peptide LLADTTHHRPWT (SEQ ID No.: 1). The results show that AVP had little effect on the three cell lines.

FIG. 8 shows digital images illustrating the localization of a fluorescently-labeled AVP phage peptide into anti-angiogenic agent responsive tumor. The right-hand images at day 17 after PBS or Bevacizumab (AVASTIN™) treatment illustrate the tumor-laden mouse in natural light.

FIG. 9 is a graph illustrating the concentration of fluorescently-labeled AVP phage peptide in a Bevacizumab (AVASTIN™)-responsive tumor, compared to the level of background (non-tumor) fluorescence in a mouse.

FIG. 10A shows digital near-infrared fluorescence images at 4 h after intravenous injection of an IRdye800-labeled scrambled peptide WTLRPTLHTDHA (SEQ ID NO.: 7) (1 nmol dye/mouse). LS174T tumor mice were imaged 1 day after a course of three doses of Bevacizumab (AVASTIN™) treatment. The scrambled peptide showed rapid renal clearance and little tumor contrast in both Bevacizumab (AVASTIN™)-treated and saline control mice.

FIG. 10B shows a graph illustrating the T/N ratios of the 2D optical images at 4 h after the administration of IRDye800-labeled scrambled peptide.

FIG. 11 shows a graph illustrating the non-responsiveness of the xenograft tumor 4T1 to Bevacizumab (AVASTIN™) therapy.

FIG. 12 illustrates a series of digital images showing that a Bevacizumab (AVASTIN™)-responsive peptide is not concentrated in a tumor (4T1) that does not respond to an anti-angiogenic therapy.

FIG. 13 illustrates a series of digital images showing that a labeled Bevacizumab (AVASTIN™)-responsive peptide is not concentrated in a 4T1 tumor (left) that does not respond to an anti-angiogenic therapy, but is concentrated in an LS174T tumor (right) that does respond to anti-angiogenic therapy, even when both types of tumor are implanted in the same animal.

FIG. 14 illustrates a series of digital images showing that a labeled Bevacizumab (AVASTIN™)-responsive peptide is not concentrated in a 4T1 tumor (left) that does not respond to an anti-angiogenic therapy, but is concentrated in a 22B tumor (right) that does respond to anti-angiogenic therapy, even when both types of tumor are implanted in the same animal.

FIG. 15 illustrates a series of photomicrographs showing the localization of a labeled Bevacizumab (AVASTIN™)-responsive peptide in tumor cells of an LS174T tumor that responds to anti-angiogenic therapy. FITC-labeled lectin is specific for the LS174T vascular endothelial cells, as is the fluorescently-labeled (Cy5.5) Bevacizumab (AVASTIN™)-responsive peptide. Brightfield and combined views are also shown.

FIG. 16 illustrates a series of photomicrographs showing the lack of localization of a labeled Bevacizumab (AVASTIN™)-responsive peptide in tumor cells of a 4T1 tumor that does not respond to anti-angiogenic therapy. FITC-labeled lectin is specific for vascular endothelial cells, unlike the fluorescently-labeled (Cy5.5) Bevacizumab (AVASTIN™)-responsive peptide that does not bind to Bevacizumab (AVASTIN™)-treated 4T1 endothelial cells. Brightfield and combined views are also shown.

FIG. 17A illustrates digital images showing a PET scan of photomicrographs showing the localization of an ¹⁸F-labeled-Bevacizumab (AVASTIN™)-responsive peptide in tumor cells of an LS174T tumor that responds to anti-angiogenic therapy.

FIG. 17B is a graph illustrating the uptake of ¹⁸F-labeled Bevacizumab (AVASTIN™)-responsive peptide in tumor cells of an LS174T tumor that responds to anti-angiogenic therapy.

FIG. 18A shows digital whole-body coronal PET images of LS174T tumor-bearing mice at 1 h and 4 h after intravenous injection of ¹⁸F-FDG (100 μCi/mouse) with or without Bevacizumab (AVASTIN™) treatment. Tumors are indicated by arrows.

FIG. 18B shows a graph illustrating that the vehicle-treated group and the Avastin-treated group had similar tumor uptake of ¹⁸F-FDG, which illustrates ¹⁸F-labeled Avastin-responsive peptide is more sensitive than ¹⁸F-FDG on detecting early tumor response to anti-angiogenetic therapy.

FIG. 19A shows digital images illustrating that an Bevacizumab (AVASTIN™)-responsive peptide is also concentrated in a tumor that responds to the anti-angiogenic drug ZD4190 therapy.

FIG. 19B is a graph illustrating the uptake of IRdye800-labelled Bevacizumab (AVASTIN™)-responsive peptide in tumor cells of a tumor that responds to the anti-angiogenic drug ZD4190 therapy.

FIG. 20A shows the digital output of gas-chromatographic analyses of ¹⁸F-labeled Bevacizumab (AVASTIN™)-responsive peptide before administering to a host animal. The structure of the ¹⁸F-labeled Bevacizumab (AVASTIN™)-responsive peptide according to the disclosure is also shown.

FIG. 20B shows the digital output of gas-chromatographic analyses of ¹⁸F-labeled Bevacizumab (AVASTIN™)-responsive peptide and in the urine thereof after 1 hr, showing minimal degradation or metabolism of ¹⁸F-labeled Bevacizumab (AVASTIN™)-responsive peptide.

FIG. 21 shows a series of digital multimodality images imaging with phage display peptide probes to monitor early anti-angiogenic therapy response. a) The optical imaging with Cy5.5 labeled AVP phage at 4 h showed no binding to a control LS174T tumor untreated tumor, but strong binding to Bevacizumab (AVASTIN™)-treated LS174T tumors. b) The optical imaging with IRDye800 labeled AVP peptide and c) microPET imaging with ¹⁸ F-labeled AVP peptide at 4 h had significantly higher uptake in the Avastin (Bevacizumab) treated tumors as compared to control PBS treated tumors.

FIG. 22 shows a series of digital images illustrating HUVEC cell uptake of Cy5.5-AVP. HUVEC only or HUVECs co-cultured with LS174T cancer cells (Transwell culture system) were treated with phosphate buffered saline (PBS) vehicle or Bevacizumab (AVASTIN™) (625 μg/ml) for 24 h, and then incubated with Cy5.5-AVP (10 nM). Shown are the photomicrographs: (A) HUVEC only, PBS; (B) HUVEC only, Bevacizumab (AVASTIN™) treatment; (C) HUVEC/LS174T co-culture, PBS; (D) HUVEC/LS174T co-culture, Bevacizumab (AVASTIN™). Only HUVEC cells co-cultured with LS174T and pre-treated with Bevacizumab (AVASTIN™) had Cy5.5-AVP uptake.

FIG. 23 is a series of schematic embodiments of the labeled probes according to the disclosure. (a) The structure of ¹⁸F-labeled Bevacizumab (AVASTIN™)-responsive peptide; (b) The anti-angiogenic therapy responsive peptide having the amino acid sequence SEQ ID NO.: 1 having a moiety (label moiety) attached thereto (a diagrammatic representation of the structre shown in (a); (c) a diagrammatic representation of a filamentous bacteriophage M13 having the anti-angiogenic therapy responsive peptide having the amino acid sequence SEQ ID NO.: 1 incorporated at one end, the peptide having a label moiety attached directly thereto; (d) a diagrammatic representation of a filamentous bacteriophage M13 having the anti-angiogenic therapy responsive peptide having the amino acid sequence SEQ ID NO.: 1 incorporated at one end, the label moiety being attached to the bacteriophage linker instead of directly to the peptide.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “ includes,” “including,” and the like; “consisting essentially of or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Abbreviations

AVP, AVASTIN™-responsive peptide; PET, positron emission tomography.

### Definitions

In describing and claiming the disclosed subject matter, the following terminology will be used in accordance with the definitions set forth below.

The term “tumor angiogenesis” as used herein refers to the ability to form new blood vessels and represents a critical step in tumor development through which the tumor establishes an independent blood supply, consequently facilitating tumor growth.

The term “angiogenesis” as used herein refers to the process of new blood vessel formation from pre-existing vessels. It plays important roles in many normal physiological functions such as embryonic development and wound healing.

The term “angiogenesis associated disease or process” as used herein refers to any disease or process that is either mediated by angiogenesis or associated with angiogenesis, including non-pathological conditions, such as blood vessel development during embryo implantation and the normal angiogenic processes in a healthy vertebrate. Angiogenesis-associated disease is a pathological condition initiated by or initiating the formation of blood vessels by endothelial cell proliferation including, but not limited to, solid tumors, blood born tumors such as leukemias; tumor metastasis; benign tumors, for example, hemangiomas, acoustic acuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulationa tumor, wet retinal macular degeneration, and the like.

The term “anti-angiogenic therapy” as used herein refers to the application of a therapeutic agent that modulates angiogenesis, e.g. by inhibiting or stimulating endothelial tube formation including the inhibition of excessive or abnormal stimulation of endothelial cells. Many molecules that inhibit tumor angiogenesis have been shown to inhibit tumor growth including antibodies against angiogenic factors such as Bevacizumab (AVASTIN™), natural and synthetic compounds that inhibit angiogenesis, and the natural angiogenic inhibitors like the angiostatin and endostatin proteins produced by tumor cells. Bevacizumab (AVASTIN™, Genentech) is a recombinant humanized monoclonal IgG1 antibody that binds to and inhibits the biologic activity of human vascular endothelial growth factor (VEGF) and which may be useful for the treatment of various malignancies. Bevacizumab (AVASTIN™) has a molecular weight of 149,000 daltons and is therefore too large to readily cross the blood-brain barrier if administered systemically. Anti-cancer therapy by inhibiting tumor angiogenesis is called anti-angiogenic therapy and has shown great potential as an effective new method for treating cancer, especially solid tumors.

The terms “anti-angiogenic therapy responsive peptide” and “peptide positively responsive” as used herein refer to a peptide, the binding of which to a vascularized tissue is modified when the vascularized target tissue is exposed to an agent that decreases the level of angiogenesis in a vascularized target tissue or other tissue. It is contemplated that the “anti-angiogenic therapy responsive peptide” binding can be induced by an antigenesis antibody such as, but not limited to Bevacizumab (AVASTIN™), or by a small molecule anti-angiogenic agent such as, but not limited to, ZD4190, a substituted 4-anilinoquinazoline inhibitor of vascular endothelial cell growth. Accordingly, the methods of the disclosure identify peptides, the binding sites of which in vascularized tissues vary in amount according to the degree of effectiveness of a therapeutic treatment intended to decrease angiogenesis in the target tissue.

The term “therapeutic agent” as used herein refers to a chemical compound useful in the treatment of cancer and which may benefit from the directed delivery to a tumor and which may be conjugated to an anti-angiogenic therapy responsive peptide or a bacteriophage delivery system according to the present disclosure. Examples of chemotherapeutic agents include alkylating agents such as thiotepa and CYTOXAN™, cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins; a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegal, dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel, ABRAXANE™, and TAXOTERE™ doxetaxel; chloranbucil; GEMZAR™ gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

The term “phage display peptide library” as used herein refers to a selection technique in which a library of variants of a peptide or protein is expressed on the outside of a phage virion, while the genetic material encoding each variant resides on the inside. This creates a physical linkage between each variant protein sequence and the DNA encoding it, which allows rapid partitioning based on binding affinity to a given target molecule (antibodies, enzymes, cell-surface receptors, etc.) by an in vitro selection process called panning. The panning is carried out by incubating a library of phage-displayed peptides with the target, washing away the unbound phage, and eluting the specifically bound phage. The eluted phage is then amplified and taken through additional binding/amplification cycles to enrich the pool in favor of binding sequences. After several rounds of panning, individual clones may be characterized by DNA sequencing and ELISA.

The term “anti-angiogenesis activity” as used herein refers to the capability of a molecule to inhibit the growth of blood vessels.

The term “positron emission tomography (PET)” as used herein refers to a nuclear medicine imaging technique that produces a three-dimensional image or map of functional processes in the body. The system detects pairs of gamma rays emitted indirectly by a positron-emitting radioisotope, which is introduced into the body on a metabolically active molecule. Images of metabolic activity in space are then reconstructed by computer analysis. Using statistics collected from tens-of-thousands of coincidence events, a set of simultaneous equations for the total activity of each parcel of tissue can be solved by a number of techniques, and a map of radioactivities as a function of location for parcels or bits of tissue may be constructed and plotted. The resulting map shows the tissues in which the molecular probe has become concentrated. Radioisotopes used in PET scanning are typically isotopes with short half lives such as carbon-11 (about 20 min), nitrogen-13 (about 10 min), oxygen-15 (about 2 min), and fluorine-18 (about 110 min). PET technology can be used to trace the biologic pathway of any compound in living humans (and many other species as well), provided it can be radiolabeled with a PET isotope. The half life of fluorine-18 is long enough such that fluorine-18 labeled radiotracers can be manufactured commercially at an offsite location.

The term “linker” as used herein refers to any molecular structure that can conjugate a peptide of the disclosure, and a moiety such as a small-molecule. The term “linker” as used herein may also include such as a bacteriophage that both expresses a peptide exposed at the head region of the phage and has a detectable marker attached thereto and which, therefore, indirectly detectably labels the peptide.

The term “radioactive label (radiolabel)” as used herein refers to a moiety conjugated to a peptide of the present disclosure wherein the moiety includes or consists of a radiolabel. Advantageous for the peptides of the disclosure are moieties that may be attached to a linker including, but not limited to, a benzoate derivative. A prosthetic group may have a radiolabel attached thereto. For example, a particularly useful prosthetic group is fluorobenzoate, wherein the carboxyl group of the benzoate may be conjugated to the γ-amino group of a glutamate linker, and the fluoride is the isotope ¹⁸F detectable by such as PET.

Some exemplary embodiments of elements that can be used as labels in the present disclosure include, but are not limited to, F-19 (F-18), C-12 (C-11), I-127 (I-125, I-124, I-131, I-123), CI-36 (CI-32, CI-33, CI-34), Br-80 (Br-74, Br-75, Br-76, Br-77, Br-78), Re-185/187 (Re-186, Re-188), Y-89 (Y-90, Y-86), Lu-177, and Sm-153, as well as those described in the figures. Imaging probes for use in the probes of the present disclosure are labeled with one or more radioisotopes, preferably including, but not limited to, ¹¹C, ¹⁸F, ⁷⁶Br, ¹²³I, ¹²⁴I, or ¹³¹I, and are suitable for use in peripheral medical facilities and PET clinics. In particular embodiments, the PET isotope can include, but is not limited to, ^{64/61}Cu, ¹²⁴I, ^{76/77}Br, ⁸⁶Y, ⁸⁹Zr, and ⁶⁸Ga.

The term “cell or population of cells” as used herein refers to an isolated cell or plurality of cells excised from a tissue or grown in vitro by tissue culture techniques. Most particularly, a population of cells refers to cells in vivo in a tissue of an animal or human.

The term “contacting a cell or population of cells” as used herein refers to delivering a peptide or a peptide-bearing bacteriophage probe according to the present disclosure to an isolated or cultured cell or population of cells or administering the probe in a suitable pharmaceutically acceptable carrier to the target tissue of an animal or human. Administration may be, but is not limited to, intravenous delivery, intraperitoneal delivery, intramuscularly, subcutaneously or by any other method known in the art. One advantageous method is to deliver directly into a blood vessel leading immediately into a target organ or tissue such as a prostate, thereby reducing dilution of the probe in the general circulatory system.

The term “pharmaceutically acceptable carrier” as used herein refers to a diluent, adjuvant, excipient, or vehicle with which a heterodimeric probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the heterodimeric probe and pharmaceutically acceptable carriers can be sterile. Water is a useful carrier when the heterodimeric probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “target” as used herein refers to a peptide, cell, tissue, tumor, etc, for which it is desired to detect. The target peptide may be on a cell surface, the cell being isolated from an animal host, a cultured cell or a cell or population of cells in a tissue of an animal.

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

The term “fluorophore” as used herein refers to a component of a molecule that causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Fluorescein isothiocyanate (FITC), a reactive derivative of fluorescein, has been one of the most common fluorophores chemically attached to other, non-fluorescent molecules to create new fluorescent molecules for a variety of applications. Other historically common fluorophores are derivatives of rhodamine (TRITC), coumarin, and cyanine. Newer generations of fluorophores such as the ALEXA FLUORS™ and the DYLIGHT FLUORS™ are generally more photostable, brighter, and less pH-sensitive than other standard dyes of comparable excitation and emission.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HILYTE™ Fluors (AnaSpec), and DYLITE™ Fluors (Pierce, Inc).

Embodiments of the detection system suitable for use in the practice of the embodiments of the disclosure includes, but is not limited to, a light-tight module and an imaging device disposed in the light-tight module. The imaging device can include, but is not limited to, a CCD camera and a cooled CCD camera.

The terms “tumor” and “cancer”, as used herein shall be given its ordinary meaning and is a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. Many tumors will also induce the formation of vascular tissue for growth and maintenance requirements of the tumor, and which provide a means of metastatic dispersal. Such vascularized tumors may be treated using anti-angiogenic compounds that inhibit the formation of vascular vessels.

Representative cancers include, but are not limited to, bladder cancer, breast cancer, colorectal cancer, endometrial cancer, head & neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, liver cancer, medulloblastoma, neuroblastoma, brain tumors generally, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, esophageal cancer, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth) and continue their rapid growth and division in this new location. This is called metastasis. Once malignant cells have metastasized, achieving cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

The term “peptide” as used herein refers to proteins and fragments thereof. Peptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term “variant” refers to a peptide or polynucleotide that differs from a reference peptide or polynucleotide, but retains essential properties. A typical variant of a peptide differs in amino acid sequence from another, reference peptide. Generally, differences are limited so that the sequences of the reference peptide and the variant are closely similar overall and, in many regions, identical. A variant and reference peptide may differ in amino acid sequence by one or more modifications (e.g., substitutions, additions, and/or deletions). A variant of a peptide includes conservatively modified variants. A substituted or inserted amino acid residue may or may not be one encoded by the genetic code. A variant of a peptide may be naturally occurring, such as an allelic variant, or it may be a variant that is not known to occur naturally.

Modifications and changes can be made in the structure of the peptides of this disclosure and still obtain a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines that peptide's biological functional activity, certain amino acid sequence substitutions can be made in a peptide sequence and nevertheless obtain a peptide with like properties.

In making such changes, the hydropathic index of amino acids can be considered. The importance of the hydropathic amino acid index in conferring interactive biologic function on a peptide is generally understood in the art. It is known that certain amino acids can be substituted for other amino acids having a similar hydropathic index or score and still result in a peptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant peptide, which in turn defines the interaction of the peptide with other molecules, such as enzymes, substrates, receptors, antibodies, antigens, and the like. It is known in the art that an amino acid can be substituted by another amino acid having a similar hydropathic index and still obtain a functionally equivalent peptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent peptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent peptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu).

As used herein, the terms “subject”, “host”, or “organism” include humans, mammals (e.g., cats, dogs, horses, etc.), living cells, and other living organisms, as well as samples such as tissue taken from a host or organism. A living organism can be as simple as, for example, a single eukaryotic cell or as complex as a mammal.

The term “detectably labeled” as used herein refers to a peptide or such as a bacteriophage comprising a peptide and containing a moiety that is radioactive, or that is substituted with a fluorophore, or that is substituted with some other molecular species that elicits a physical or chemical response that can be observed or detected by the naked eye or by means of instrumentation such as, without limitation, scintillation counters, colorimeters, UV spectrophotometers and the like. As used herein, a “label” or “tag” refers to a molecule that, when appended by, for example, without limitation, covalent bonding or hybridization, to another molecule, for example, also without limitation, a peptide, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter.

Suitable labeling moieties may be, for example, a radiolabel (e.g., ³H, ¹²⁵I, ³⁵S, ¹⁴C, ³²P, etc.), a fluorescent dye (such as, but not limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.,), HILYTE™ Fluors (AnaSpec), and DYLITE™ Fluors (Pierce, Inc), or any other moiety capable of generating a detectable signal such as a colorimetric, fluorescent, chemiluminescent or electrochemiluminescent (ECL) signal.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g. on the same, or a neighboring molecule).

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme Qβ replicase and a ribonucleic acid (RNA) sequence template attached to a probe complementary to the DNA to be copied which is used to make a DNA template for exponential production of complementary RNA; strand displacement amplification (SDA); Qβ replicase amplification (QβRA); self-sustained replication (3SR); and NASBA (nucleic acid sequence-based amplification), which can be performed on RNA or DNA as the nucleic acid sequence to be amplified.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

The terms “peptide” or “protein” as used herein are intended to encompass a protein, a glycoprotein, a peptide, a peptide, and the like, whether isolated from nature, of viral, bacterial, plant, or animal (e.g., mammalian, such as human) origin, or synthetic, and fragments thereof.

The term “nucleic acid” as used herein refers to DNA and RNA, whether isolated from nature, of viral, bacterial, plant or animal (e.g., mammalian, such as human) origin, synthetic, single-stranded, double-stranded, comprising naturally or non-naturally occurring nucleotides, or chemically modified.

The term “expressed” or “expression” as used herein refers to the transcription from a gene to give an RNA nucleic acid molecule at least complementary in part to a region of one of the two nucleic acid strands of the gene. The term “expressed” or “expression” as used herein can also refer to the translation of RNA to produce a protein or peptide.

The term “phage display library” as used herein especially applies to the The Ph.D.™-12 Phage Display Peptide Library (New England Biolabs) which, according of the manufacturer's information “is based on a combinatorial library of random dodecapeptides fused to a minor coat protein (pIII) of M13 phage. The displayed peptide (12-mer) is expressed at the N-terminus of pIII, i.e., the first residue of the mature protein is the first randomized position. The peptide is followed by a short spacer (Gly-Gly-Gly-Ser) and then the wild-type pIII sequence. The library consists of approximately 2.7×10⁹ electroporated sequences amplified once to yield approximately 100 copies of each sequence in 10 μl of the supplied phage. M13 is a filamentous bacteriophage composed of circular single stranded DNA 6407 nucleotides long encapsulated in approximately 2700 copies of the major coat protein P8, and capped with 5 copies of two different minor coat proteins (P9, P6, P3) on the ends. The minor coat protein P3 attaches to the receptor at the tip of the F pilus of the host Escherichia coli. Infection with filamentous phages is not lethal, however the infection causes turbid plaques in E. coli. It is a non-lytic virus.

### Discussion

Rapid assessment of a cancer response to a therapeutic regimen can determine efficacy early in the course of treatment. The present disclosure provides a class of molecular imaging probes that can indicate tumor early responses to anti-angiogenic therapies, such as that based on, but not limited to, Bevacizumab (AVASTIN™). The compositions of the present disclosure are further contemplated as suitable for use as delivery vehicles for the directed delivery of small molecules such as, but not limited to, therapeutic agents to a tissue that can selectively bind an anti-angiogenic therapy responsive peptide according the present disclosure.

Tumor vascular beds in different phases of anti-angiogenic treatment are morphologically and functionally different (Jain R. K. (2001) Nat. Med. 7: 987-989; Winkler et al., (2004) Cancer Cell 6: 553-563; Jain R. K. (2005) Science 307: 58-62; Tong et al., (2004) Cancer Res. 64: 3731-3736), which suggested changes in molecular expression, or the appearance of new molecules. The identification of specific biomarkers help to identify responsive patients and optimal doses, validate mechanistic hypotheses, predict efficacy of treatment regimens, and to detect and avoid tumor escape (Jain et al., (2006) Nat. Clin. Pract. Oncol. 3: 24-40). However, the identification of tumor vascular markers has progressed slowly, at least partially because of difficulties in isolating pure populations of endothelial cells from tumor tissues and because isolated and cultured cells may lose their tissue-specific traits upon culture (Borsum et al., (1982) Atherosclerosis 44: 367-378; Augustin et al., (1994) Bioessays 16: 901-906). The phenotype of endothelial cells is unstable and likely to change when the cells are removed from their tumor microenvironments.

The present disclosure, therefore, provides methods for the non-invasive imaging of vascularized tissues where a detectable label may be concentrated in the tissue by selective binding between an isolated and detectably labeled peptide and a site of the vascularized tissue, the level of which site can be modulated by an anti-angiogenic therapeutic treatment.

The binding of anti-angiogenic therapy responsive peptides isolated by the methods of the disclosure corresponds to the efficacy of the therapeutic treatment due to an increase in the amount of a binding site in the tissue. The data of the disclosure shows that such changes in binding site levels occurs specifically at a site of anti-angiogenic activity and is only found in tissues such as a tumor tissue responsive to anti-angiogenic therapy.

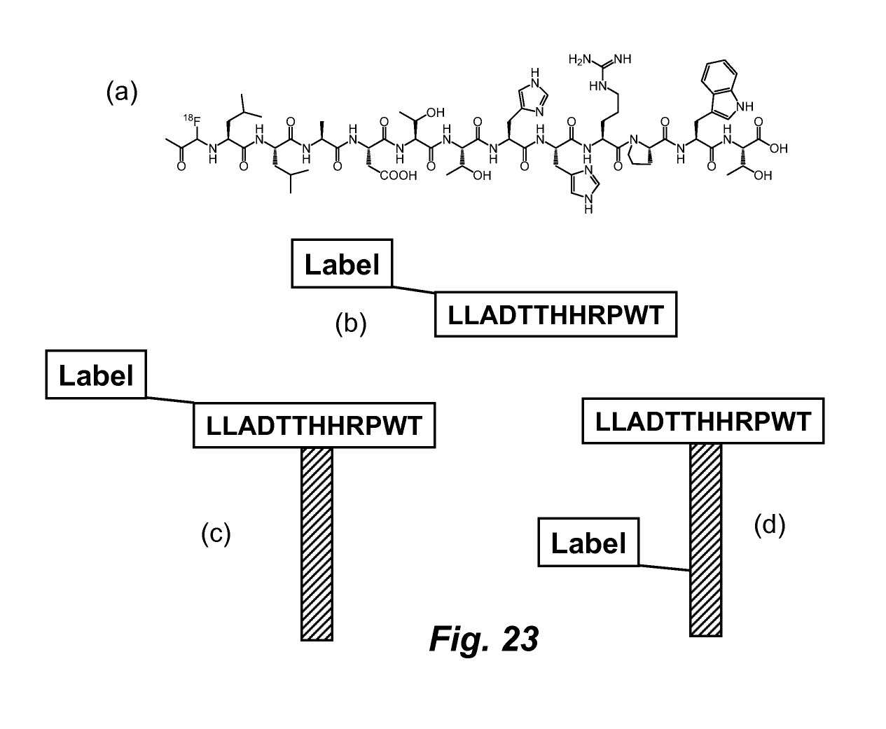

While it is considered that the anti-angiogenic therapy responsive peptides identified by the methods of the disclosure may be directly labeled for use as imaging probes, it is also considered within the scope of the disclosure for the peptides to further be attached to non-labeled moieties such as therapeutic agents for the selective delivery of such agents to the target tissue. Accordingly, it is possible by means of the peptides of the disclosure to both monitor tumor size and the efficacy of anti-angiogenic and therapeutic treatments non-invasively. It is further contemplated, as shown by the examples of the disclosure to allow the isolated peptides to be incorporated (displayed) on a bacteriophage which can then function as a delivery vehicle within recipient subject animal or host. The bacteriophage in such compositions may be a passive carrier or have one or moieties attached directly thereto (instead of or in addition to the moieties being attached to the peptide itself (se FIG. 23 for some possible embodiments thereof).

Phage display is a very useful technique to obtain defined peptide sequences that interact with a particular molecule. The application of phage display to discover tumor-homing peptides has been reported (Seung-Min et al., (2009) Methods Mol. Biol. 512: 355-363). One of the most exciting recent developments has been the use of in vivo phage display to yield disease-specific or organ-specific phage clones (Pasqualini & Ruoslahti (1996) Nature 380: 364-366; Rajotte et al., (1998) J. Clin. Invest. 102: 430-437) and phage displayed peptides recovered from irradiated tumors have also been used to assess cancer response to irradiation therapy (Han et al., (2008) Nat. Med. 14 :343-349; Hallahan et al., (2003) Cancer Cell 3: 63-74).

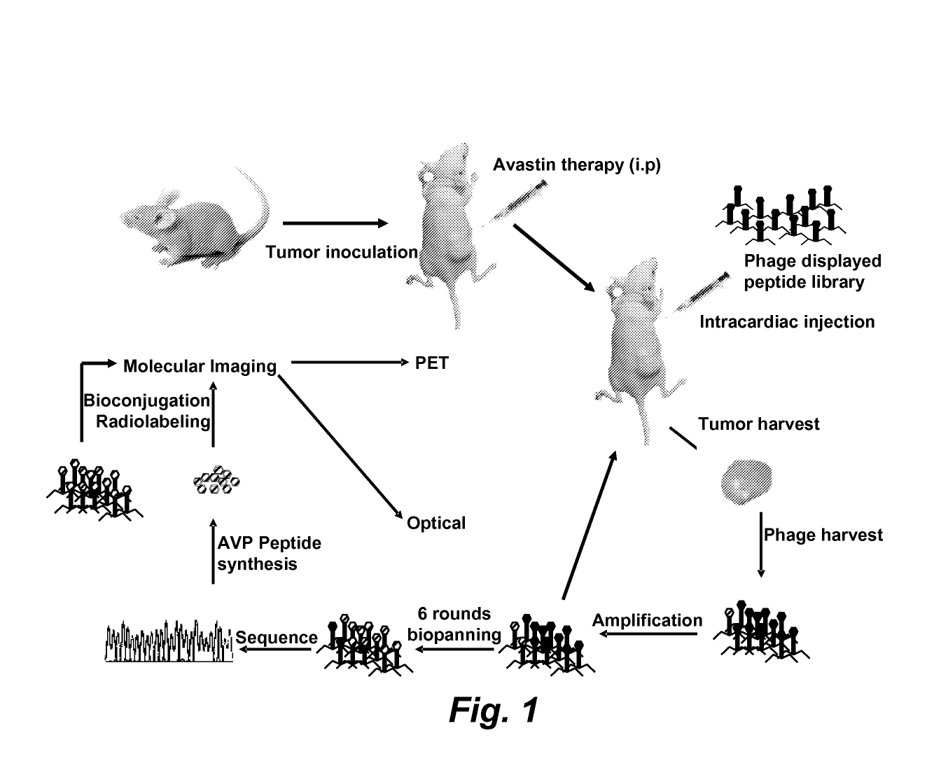

Anti-angiogenic treatment responses to Bevacizumab (AVASTIN™) were evaluated with a LS174T colorectal cancer xenograft model. A phage-displayed peptide library was bio-panned screened for peptides that can selectively bind to the cells (vascular and/or tumor cells) of a tumor responsive to an anti-angiogenic agent, as shown in FIG. 1.

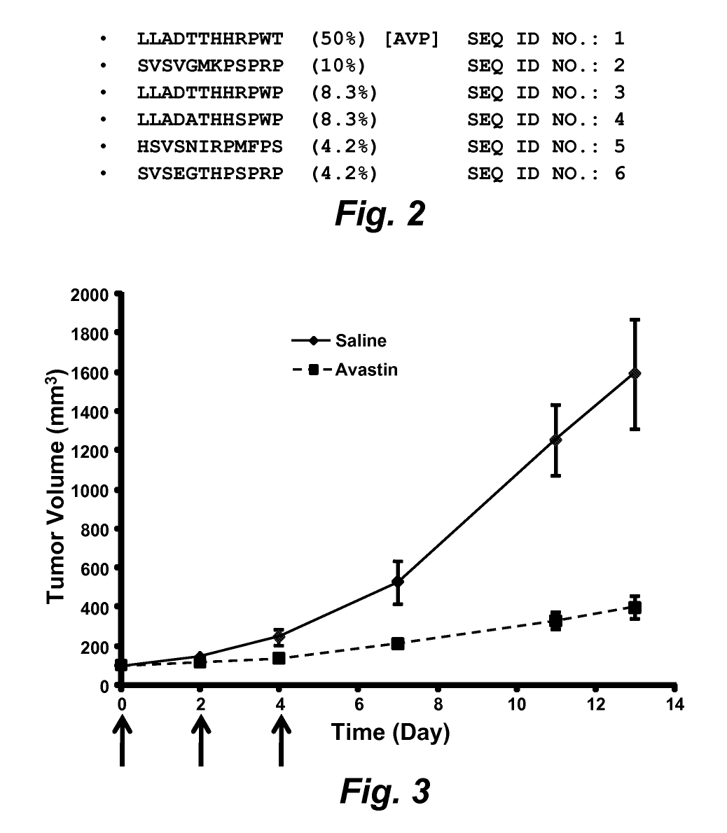

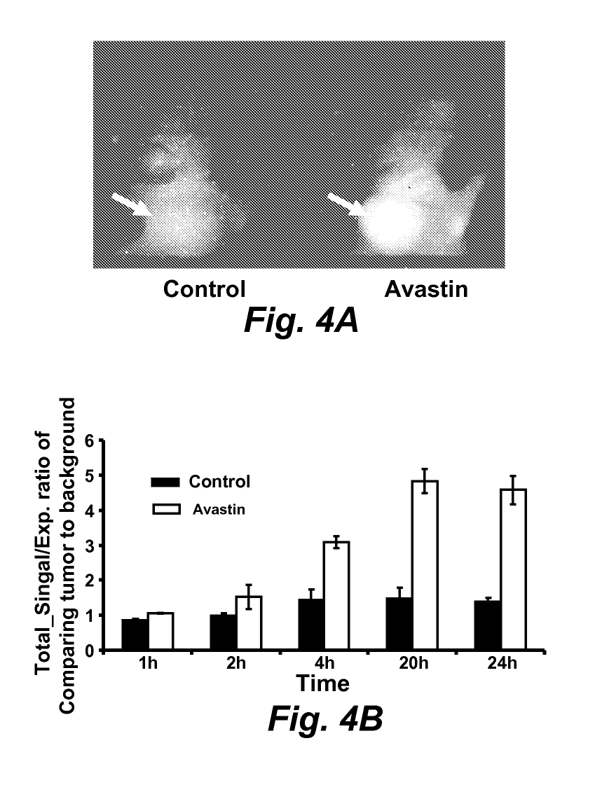

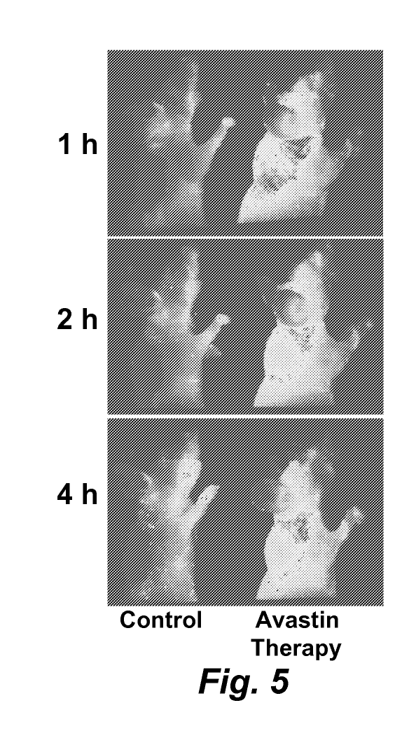

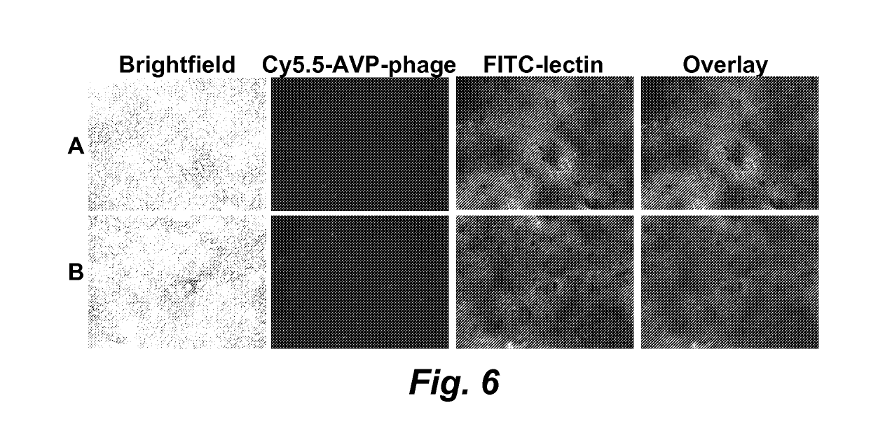

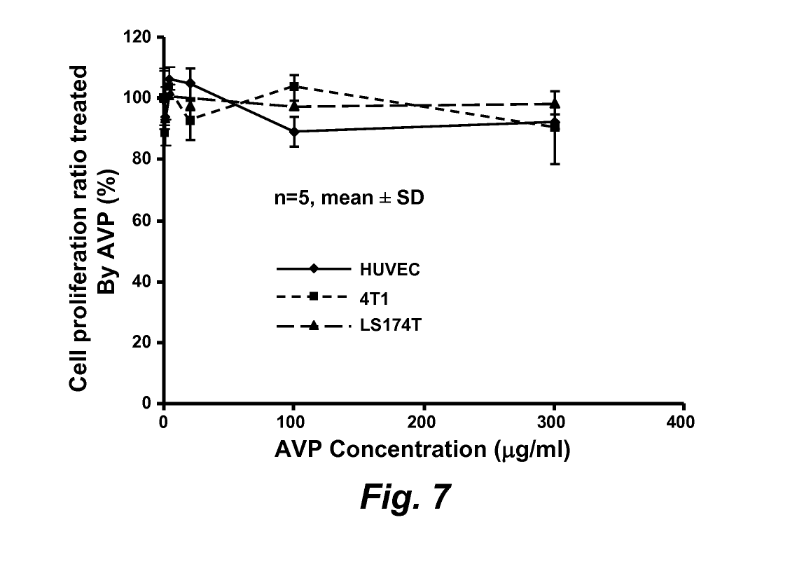

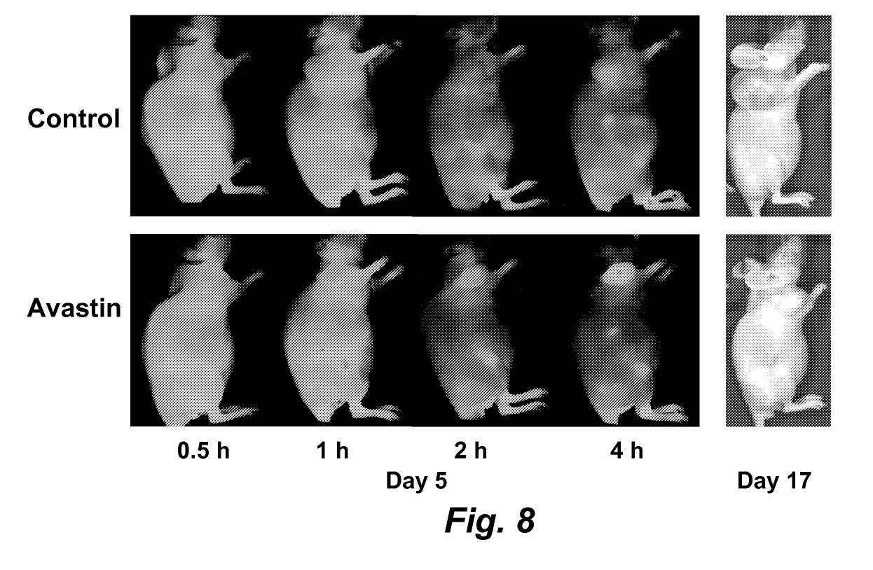

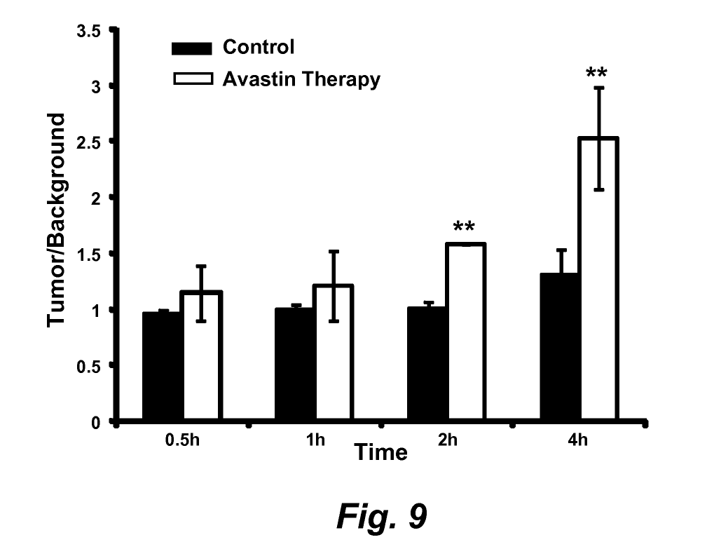

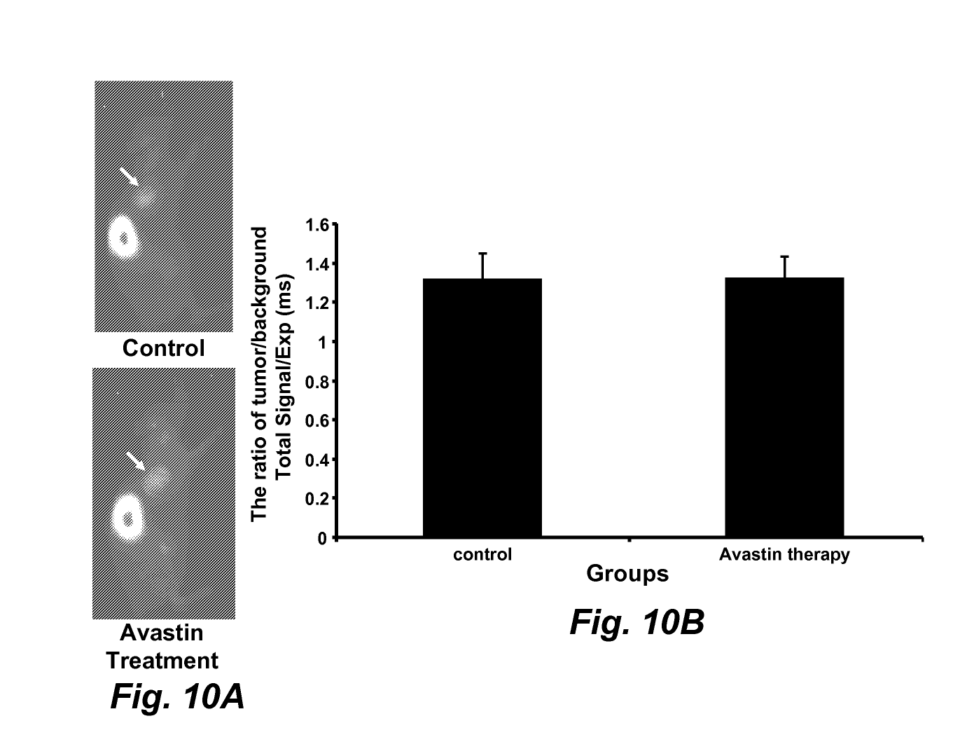

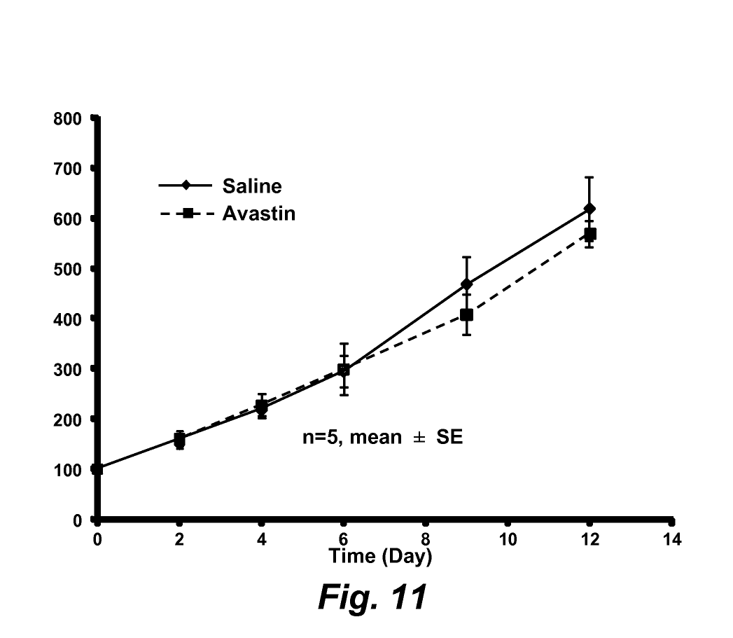

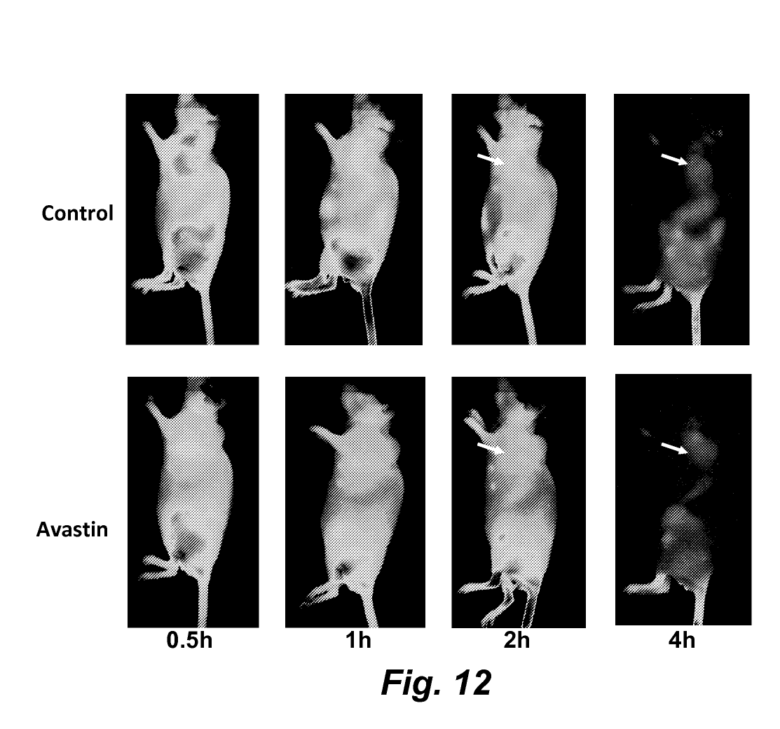

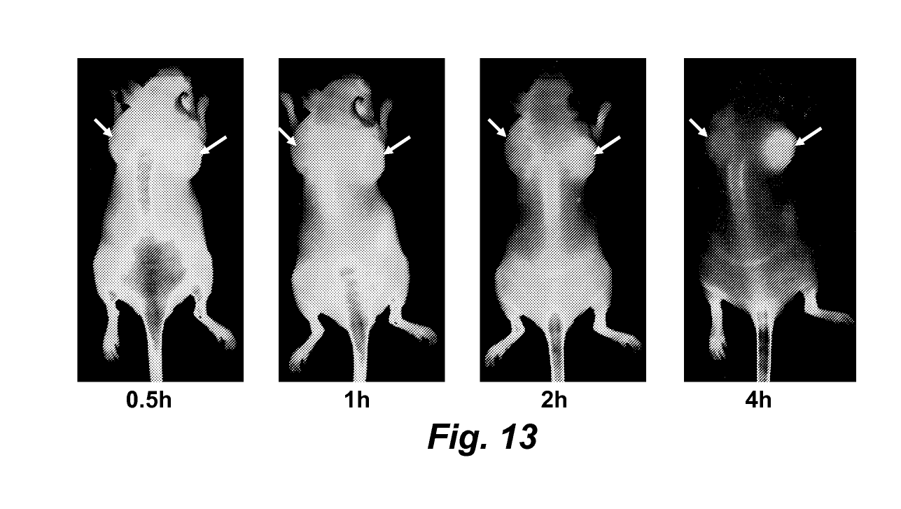

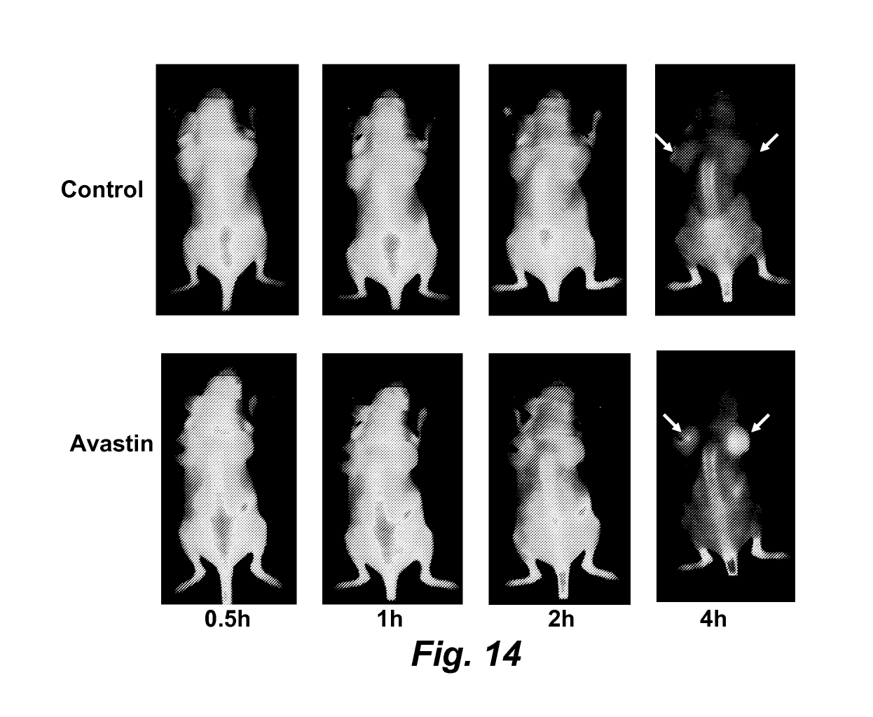

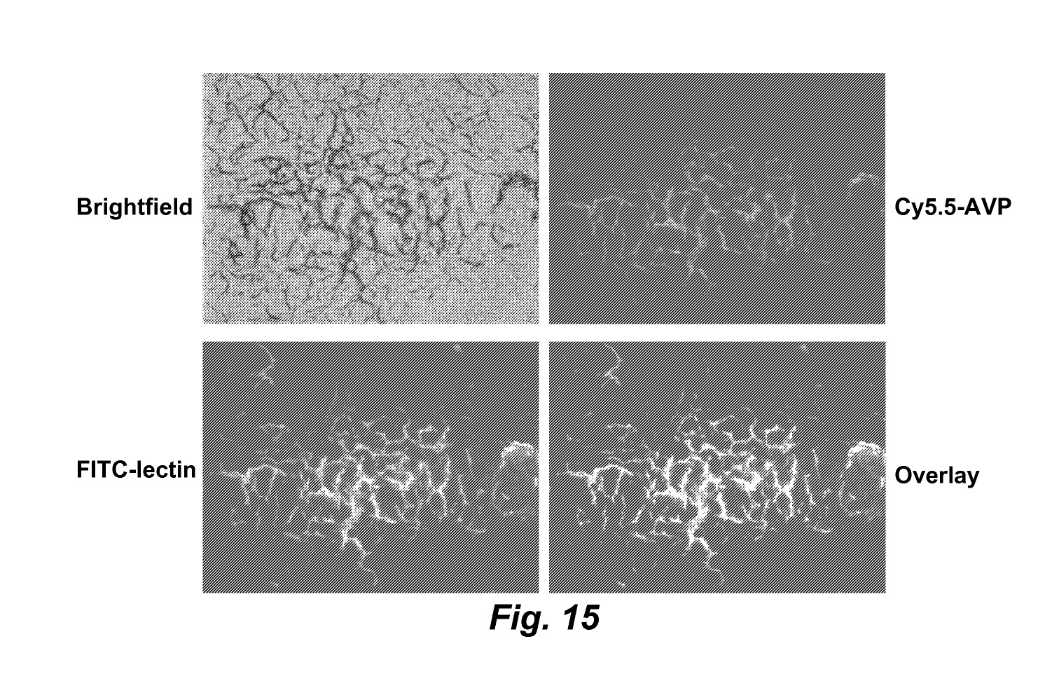

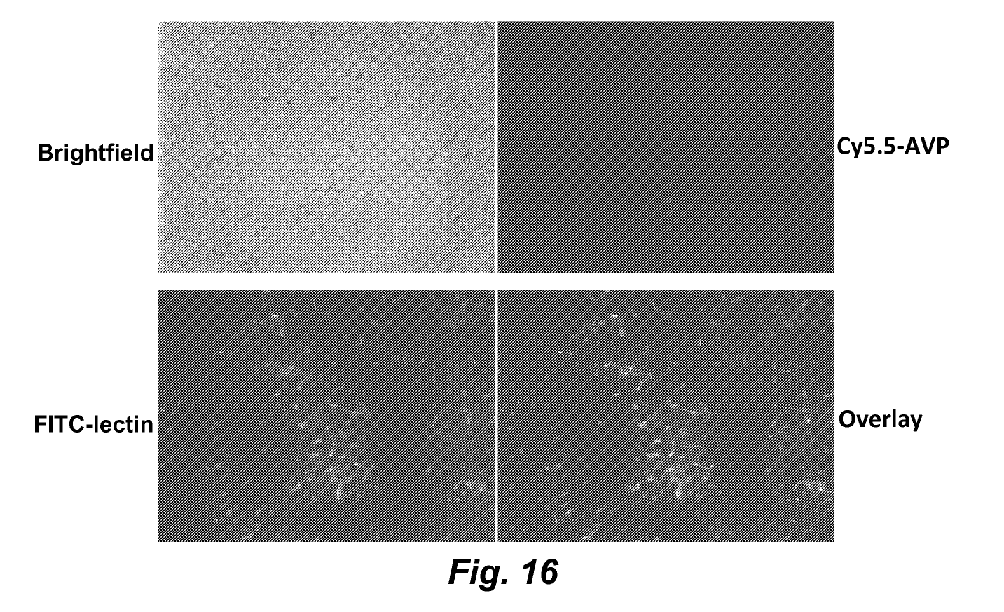

Phage-displayed peptides (as shown in FIG. 2, for example) that selectively bind to anti-angiogenic therapy-responsive cells were recovered from treated tumors. The high percentage amino acid sequences were isolated after repeated rounds of bio-panning. The amino acid sequences of the most frequently isolated peptides that had the greatest affinity for the targeted cells were determined from the nucleotide sequences of the regions of the genomes of the isolated (cloned) phages that express each of the peptides. The Bevacizumab (AVASTIN™)-responsive peptide (AVP) was evaluated by both optical and positron emission tomography (PET) imaging studies as an anti-angiogenic responsive tumor selective agent.

Accordingly, embodiments of the methods of the present disclosure encompass in vivo phage display to screen and identify a linear 12-mer phage peptide sequence (SQ ID NO.: 1) that binds specifically to tumor vascular beds subjected to effective Bevacizumab (AVASTIN™) treatment. This peptide, when coupled with fluorescent dyes such as Cy5.5 or IRDye800 for NIR fluorescence imaging, or labeled with ¹⁸F through a prosthetic labeling group ¹⁸F-2-fluoroproprionate (¹⁸F-FP), showed significantly higher accumulation in Bevacizumab (AVASTIN™)-responding LS174T and 22B tumor models, both of which secrete high levels of human VEGF. A scrambled peptide used as a control, however, showed rapid renal clearance and no tumor accumulation in LS174T tumors treated with either vehicle control or bevascizumab. Murine 4T1 tumors that do not respond to Bevacizumab (AVASTIN™) had no binding to this AVP peptide, further indicating the specificity of this peptide sequence to Bevacizumab (AVASTIN™) responsive tumors.

The change in peptide uptake preceded anatomical changes that could be measured by caliper. ¹⁸F-FDG uptake measurements, and hence glucose metabolism, failed to disclose a difference between the control and Bevacizumab (AVASTIN™)-treated 4T1 tumors tumors, implying that the viability of the tumor cells was essentially unaltered after Bevacizumab (AVASTIN™) exposure. It is of note that for both the LS174T and 22B tumor models, a difference in tumor uptake of AVP 24 h after the 3rd dose of Bevacizumab (AVASTIN™) treatment (that is the fifth day after Bevacizumab (AVASTIN™) treatment began) was not seen, even though no difference in tumor volume could be detected. Statistically significant changes in tumor volume between the Bevacizumab (AVASTIN™)-treated group and saline control group were not observed until an additional 5-6 days after the imaging studies had shown uptake of AVP. Such findings in rodents, when extrapolated to humans can be equivalent to detecting changes in tumors earlier than might be possible by using CT or MRI to detect changes in tumor size.

The data now indicate that AVP binds specifically to tumor endothelial cells exposed to Bevacizumab (AVASTIN™), but not to untreated ones. The increase of AVP binding is predictive of effective Bevacizumab (AVASTIN™) treatment, even though the exact target of this AVP sequence is still unknown. Accordingly, it has shown that the increase in AVP peptide binding during the response to therapy can distinguish between responsive and nonresponsive cancers.

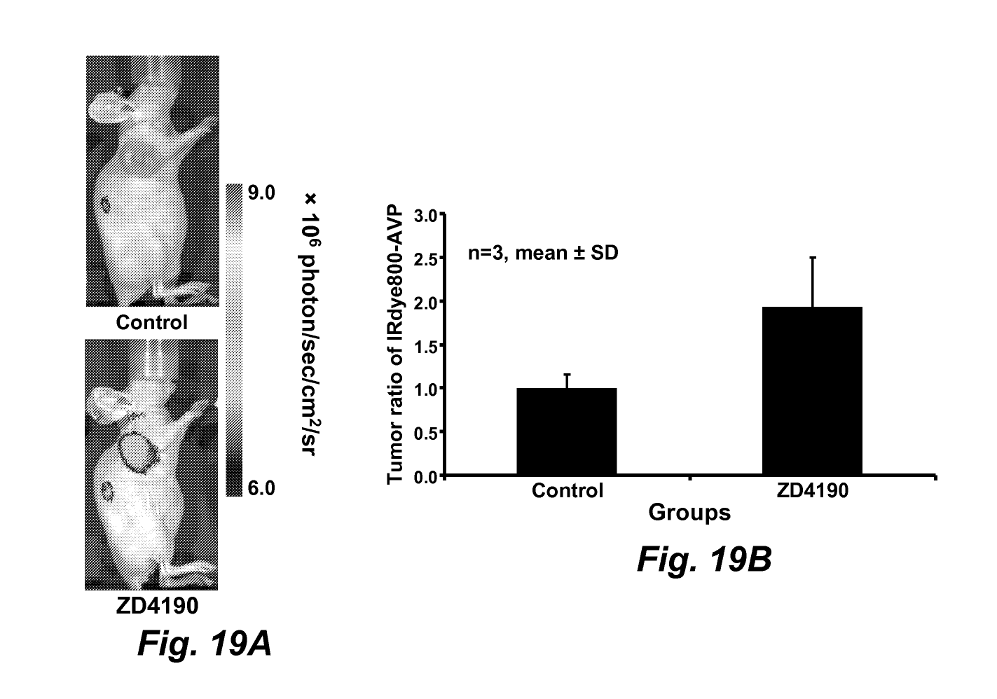

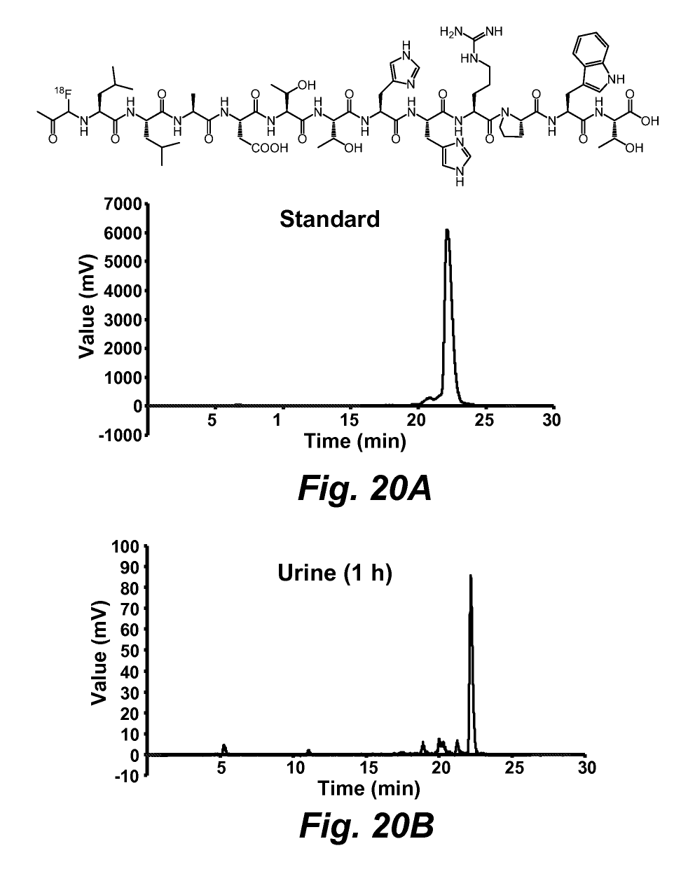

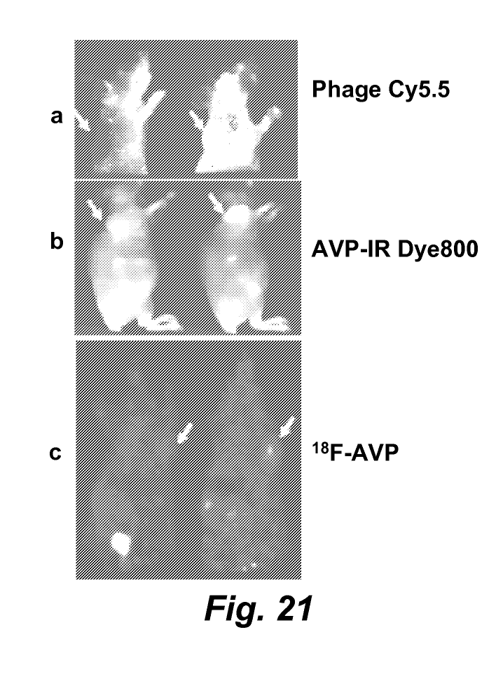

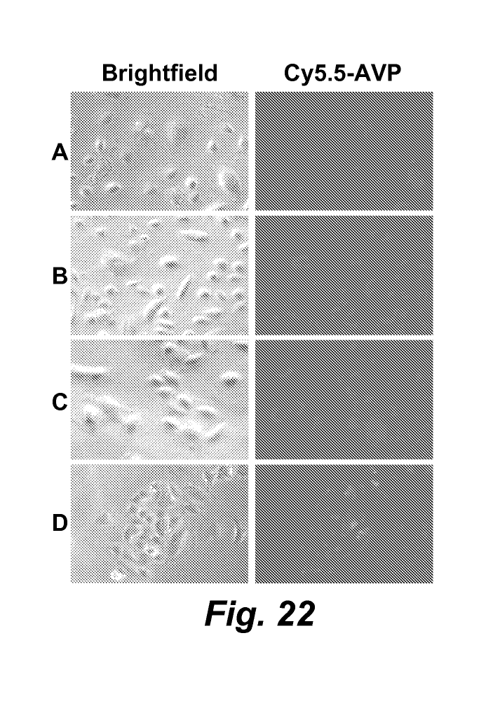

A linear 12-mer peptide, LLADTTHHRPWT (SEQ ID NO.: 1), has been identified from a phage display that, when conjugated with near-infrared fluorescent dyes or radionuclides, has the ability to distinguish between tumors that respond to Bevacizumab (AVASTIN™) and those that don't. It has also been found that an anti-angiogenic therapy responsive peptide, and in particular SEQ ID NO.: 1 that had been induced by the treatment of a vascularized tumor with Bevacizumab (AVASTIN™), will also selectively localize in tumors that are treated with a small-molecule anti-angiogenic agent, namely ZD1490, as shown in FIGS. 19A and 19B. It is, therefore, contemplated that the uses of anti-angiogenic therapy responsive peptides and the methods of the present disclosure may be applied not only to monitoring the efficacy of anti-angiogenic therapies where the agent is such as an antibody directed to vascular endothelial cells or other factors generating the tumor vascular system, but may also be applied where the anti-angiogenic therapeutic agent is a small-molecule agent. The rapid, non-invasive assessment of pharmacodynamic responses by using peptides such as disclosed herein promise to accelerate drug development and to allow earlier discontinuation of ineffective treatments.

Accordingly, the present disclosure encompasses peptides selected using phage display techniques that allow the visualization of early tumor responses to anti-angiogenic treatment. A suitably labeled anti-angiogenic-responsive specific peptide such as the AVP peptide is useful for monitoring treatment responses. It is contemplated that such peptides may be labeled by direct conjugation of a detectable moiety to the peptide, conjugated via a linker molecule, oligopeptide linker, or the like, or by expressing the peptide on the surface of a bacteriophage, which may then be labeled.

The detectable moieties that may be suitable for use in labeling the peptides of the present disclosure include any that are applicable to whole-body scanning of host animals, including fluorescent labels, labels suitable for positron-emission tomography (PET scanning) and the like. It is further within the scope of the disclosure for more than one type of label to be conjugated to the peptide agents to allow for more than one scanning system to be used to detect the tumor.

One aspect of the present disclosure, therefore, provides embodiments of a method for identifying an anti-angiogenic therapy-responsive peptide, comprising: (a) providing a subject animal or human having a target tissue responsive to an anti-angiogenic therapeutic agent; (b) administering an anti-angiogenic therapeutic agent to the subject animal or human; (c) delivering to the subject animal or human a phage-displayed peptide library under conditions allowing at least one bacteriophage species from the phage-displayed peptide library to selectively bind to an anti-angiogenic therapy-responsive site of the target tissue; (d) isolating the target tissue from the subject animal or human; (e) isolating from the tumor a population of bacteriophages; (f) amplifying said population of bacteriophages in a bacterial host and harvesting said amplified bacteriophages; (g) repeating steps (b)-(f), thereby enriching the isolated phage population for peptide-bearing phage comprising an anti-angiogenic therapy-responsive peptide; and (h) determining the nucleotide sequence encoding a phage-displayed peptide isolated by steps (a)-(g), thereby identifying a peptide positively responsive to an anti-angiogenic therapy.

In embodiments of this aspect of the disclosure, the tissue can be a pathological tissue. In some embodiments of this aspect of the disclosure, the pathological tissue is a tumor.

In some embodiments of this aspect of the disclosure, the anti-angiogenic therapeutic agent may comprise an antibody or a fragment thereof.

In one embodiment of this aspect of the disclosure, the anti-angiogenic therapeutic agent may be, but is not limited to, bevacizmuab (AVASTIN™).

Another aspect of the present disclosure encompasses isolated bacteriophages comprising an anti-angiogenic therapy-responsive peptide, wherein the isolated bacteriophage is characterized as selectively binding to a vascularized target tissue responsive to an anti-angiogenic therapeutic agent, or cells isolated therefrom.

In embodiments of this aspect of the disclosure, the vascularized target tissue can be a pathological tissue.

In embodiments of this aspect of the disclosure, the pathological tissue can be a tumor.

In embodiments of this aspect of the disclosure, the anti-angiogenic therapy-responsive peptide may comprise an amino acid sequence selected from the group consisting of: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, where said peptide is characterized as having selective affinity for a vascularized target tissue responsive to an anti-angiogenic therapeutic agent, or cells isolated therefrom.

In some embodiments of this aspect of the disclosure, the anti-angiogenic therapy-responsive peptide can comprise the amino acid sequence selected from the group consisting of: LLADTTHHRPWT (SEQ ID NO.: 1), or a conservative variant thereof.

In some embodiments of this aspect of the disclosure, the anti-angiogenic therapy-responsive peptide may comprise the amino acid sequence selected from the group consisting of: LLADTTHHRPWT (SEQ ID NO.: 1).

In embodiments of this aspect of the disclosure, the isolated bacteriophage can further comprise a moiety or plurality of moieties attached to the bacteriophage and selected from the group consisting of: a therapeutic agent, a detectable label, and a combination thereof. In embodiments of this aspect of the disclosure, the detectable label can be selected from the group consisting of: a fluorescent label, a PET detectable label, an MRI-detectable label, and a radioactive label.

Yet another aspect of the disclosure provides isolated anti-angiogenic therapy-responsive peptides that can comprise an amino acid sequence selected from the group consisting of: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, where the peptide is characterized as having selective affinity for a site of a vascularized target tissue responsive to an anti-angiogenic therapeutic agent, or cells isolated therefrom.

In these embodiments, the tissue can be a pathological tissue such as a tumor.

In some embodiments, the anti-angiogenic therapy-responsive peptide can have the amino acid sequence SEQ ID NO.: 1.

In embodiments of this aspect of the disclosure, the anti-angiogenic therapy-responsive peptides can further comprise a moiety or plurality of moieties attached to the bacteriophage and selected from the group consisting of: a therapeutic agent, a detectable label, and a combination thereof. In some of these embodiments, the detectable label can be selected from the group consisting of: a fluorescent label, a PET detectable label, an MRI-detectable label, and a radioactive label.

Still another aspect of the disclosure provides compositions selective for an anti-angiogenic therapy-responsive tissue in a subject animal or human comprising an anti-angiogenic therapy-responsive peptide linked to a moiety or plurality of moieties desired to be delivered to a tissue characterized as selectively binding anti-angiogenic therapy-responsive peptide, where the peptide is characterized as having selective affinity for a vascularized tissue responsive to an anti-angiogenic therapeutic agent, or cells isolated therefrom.

In these embodiments of this aspect of the disclosure, the vascularized tissue can be a pathological tissue such as, but not limited to, a tumor.

In embodiments of this aspect of the disclosure, the compositions can further comprise a bacteriophage having anti-angiogenic therapy-responsive peptide expressed thereon, and wherein the moiety or plurality of moieties desired to be delivered to a tissue is optionally attached to the peptide or to the bacteriophage.

In embodiments of the compositions of this aspect of the disclosure, the anti-angiogenic therapy-responsive peptide can be selected from the group consisting of the amino acid sequences: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof.

In some embodiments of the compositions of this aspect of the disclosure, the anti-angiogenic therapy-responsive peptide has the amino acid sequence LLADTTHHRPWT (SEQ ID NO.: 1), or a conservative variant thereof.

In certain embodiments of the compositions of this aspect of the disclosure, the anti-angiogenic therapy-responsive peptide has the amino acid sequences: LLADTTHHRPWT (SEQ ID NO.: 1).

In embodiments of the compositions of this aspect of the disclosure, the moiety or plurality of moieties desired to be delivered to a vascularized target tissue and linked to the anti-angiogenic therapy-responsive peptide can be selected from the group consisting of: a therapeutic agent, a detectable label, and a combination thereof.

In some embodiments of the compositions of this aspect of the disclosure, the detectable label can be selected from the group consisting of: a fluorescent label, a PET detectable label, an MRI-detectable label, and a radioactive label.

In embodiments of the compositions of this aspect of the disclosure, the compositions may further comprise a pharmaceutically acceptable carrier.

Yet another aspect of the disclosure provides embodiments of methods of imaging a tissue in an animal or human subject, comprising: (a) delivering to a subject animal or human a pharmaceutically acceptable composition comprising an anti-angiogenic therapy-responsive peptide linked to a detectable label, where the anti-angiogenic therapy-responsive peptide is characterized as selectively concentrating at a site in a vascularized target tissue; and (b) detecting the label in the subject animal or human, thereby identifying the location of an anti-angiogenic therapy-responsive vascularized target tissue in the host.

In embodiments of this aspect of the disclosure, the target tissue can be a pathological tissue, and in certain embodiments the pathological tissue can be a tumor.

In embodiments of this aspect of the disclosure, the anti-angiogenic therapy-responsive peptide is selected from the group consisting of the amino acid sequences: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, wherein said peptide is characterized as having selective affinity for a vascularized tumor responsive to an anti-angiogenic therapeutic agent, or cells isolated therefrom.

In some embodiments of this aspect of the disclosure, the anti-angiogenic therapy-responsive peptide has the amino acid sequence LLADTTHHRPWT (SEQ ID NO.: 1), or a conservative variant thereof.

In some embodiments of this aspect of the disclosure, the anti-angiogenic therapy-responsive peptide has the amino acid sequences: LLADTTHHRPWT (SEQ ID NO.: 1).

In embodiments of the methods of this aspect of the disclosure, the anti-angiogenic therapy-responsive peptide can be expressed by a bacteriophage and the detectable label is attached to the bacteriophage. In these embodiments the detectable label can be selected from the group consisting of: a fluorescent label, a PET detectable label, an MRI-detectable label, and a radioactive label.

In some embodiments of this aspect of the disclosure, the method can further comprise the steps of: (i) delivering to the subject a therapeutic agent; (ii) periodically imaging the vascularized target tissue in the subject; and (iii) determining from the image size of the tissue whether the therapeutic agent is effective in reducing the size of the tumor in the subject.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### Examples

### Example 1

Cell Lines: The tumor cell lines (LS174T, 22B and 4T1) were purchased from American Type Culture Collection (ATCC) and were maintained in medium supplemented with 10% FCS and 1% penicillin-streptomycin as ATCC recommends. Normal Human Umbilical Vein Endothelial Cells (HUVECs) and relevant culture medium were purchased from PromoCell (Germany).

### Example 2

Chemicals: Bevacizumab (AVASTIN™) was purchased from Genentech/Roche. IRdye800-NHS and Cy5.5-NHS were from Li-Cor and GE Healthcare, respectively. Fluorescein isothiocyanate (FITC)-labeled tomato lectin was from Thermo Fisher Scientific (Rockford, Ill.). The AVP peptide was synthesized by Peptides International.

### Example 3

Animal Models: Female athymic nude mice (nu/nu) were obtained from Harlan (Indianapolis, Ind.) at 6-8 weeks of age and were kept under sterile conditions. The LS174T or 22B cells were harvested and suspended in sterile PBS at a concentration of 5×10⁷ viable cells/ml. Viable tumor cells (5×10⁶) in sterile PBS (100 μL) were injected subcutaneously into the right shoulder. The 4T1 cells were harvested from the tumor and suspended in sterile PBS at a concentration of 2×10⁷ viable cells/mL. Viable cells (2×10⁶) in sterile PBS (100 μL) were injected subcutaneously into the left shoulder. Tumor growth was followed by caliper measurements of perpendicular measures of the tumor. The tumor volume was estimated by the formula: tumor volume=a×(b2)/2, where a and b were the tumor length and width respectively in mm.

### Example 4

Tumor Growth Study: When palpable tumors (150-200 mm³) were present in all animals, mice were randomly divided into two groups (n=10/group). Cancer therapy response was evaluated in LS174T human colorectal cancer, 4T1 murine breast cancer and 22B human head-neck cancer models. The mice were injected intraperitoneally with 20 mg/kg of Bevacizumab (AVASTIN™) every other day for a total of three doses. The mouse body weight and tumor volume were measured every 3 days for up to 20 days before euthanasia.

### Example 5

Biopanning Phage-Displayed Libraries: Biopanning was conducted in vivo with phage displayed peptide libraries (Ph.D.™-12 phage display peptide library, New England Biolabs

Inc.). The phage displayed peptide library represented 1×10⁹ independent clones of phages expressing random 12-mer peptides that are displayed on M13 phages. After the tumor-bearing mice were treated, phage libraries were administered by intracardiac injection. The amplified phages were partially purified by polyethyleneglycol (PEG) precipitation and resuspended in tris buffered saline (TBS) for the next round of biopanning.

A schema for the identification of Avastin responsive peptide (AVP) is shown in FIG. 1. LS174T tumor-bearing mice were treated with 3 doses of Bevacizumab (AVASTIN™) (intraperitoneal injection at 20 mg/kg every other day). A Ph.D.™-12 phage display peptide library expressing random 12-mer peptides on M13 phages were injected intracardiacally into the mice 1 day after the third dose of Bevacizumab (AVASTIN™) treatment. Mice were sacrificed 10 min after phage injection, and the tumors were harvested. The particles were collected and amplified for the next round of biopanning. After six rounds of biopanning, single plaques from soft agar were isolated. The peptide sequences were deduced from the decoded DNA information. The AVP peptide was then appropriately labeled for optical and positron emission tomography (PET) imaging.

### Example 6

Phage Labeling: Phages were labeled with a near-infrared dye IRdye800-NHS or Cy5.5. Phages (1×10¹² pfu) were resuspended in 100 μl of 0.3 M sodium bicarbonate (pH 8.6) solution containing 0.1 mg/ml fluorochrome-hydroxy-succinimide ester. The phage/fluorochrome reaction was allowed to continue for 1 h at room temperature in the dark. The volume of the labeled phage was then brought up to 1 ml with Dulbecco's Phosphate Buffered Saline (DPBS), and the phage was purified by PEG precipitation. Fluorochrome-labeled phage was then resuspended in 200 μl of DPBS and titered to determine plaque-forming units, and the concentration of fluorochrome was determined spectrophotometrically (Kelly et al., (2006) Neoplasia 8: 1011-1018).

### Example 7

Metabolic Stability of AVP Peptide: Nude mice bearing LS174T tumor xenografts were intravenously injected with 3.7 MBq of ¹⁸F-FP-AVP. Urine samples were collected 1 h after tracer injection and analyzed by HPLC.

Metabolic stability of AVP peptide: (A) Analytical HPLC chromatogram of ¹⁸F-FP-AVP standard. Pure ¹⁸F-FP-AVP had a retention time of about 22 min following the conditions described in methods. (B) HPLC of urine sample at 1 h after injection of ¹⁸F-FP-AVP showed about 90% intact tracer, with several minor peaks with different retention times, suggesting good metabolic stability of this linear 12-mer peptide in vivo.

### Example 8

Toxicity of AVP on Cell Viability by MTT Assay: MTT (3-(4,5-dimethylthiazolyl-2)-2,5-diphenyltetrazolium bromide; ATCC) assays were used to measure cell viability. Three thousand tumor cells or HUVECs were seeded per well in a 96-well plate and allowed to incubate for 24 h. After incubation with various concentrations of peptide AVP for 48 h, 10 μl of MTT reagent was added to each well. Four hours later, when the purple precipitate became visible, the supernatant was discarded, and 100 μl of DMSO was added to each well and the plate was shaken in the dark for 10 min at room temperature. The absorbance at 570 nm was then measured using a microplate reader (Tecan).

### Example 9

Near-Infrared Fluorescence Imaging: The tumor-bearing mice (with or without Bevacizumab (AVASTIN™) treatment) were injected intravenously with appropriately labeled phages or AVP peptide (1 nmol dye/mouse). Two-dimensional NIR fluorescence images were acquired at various time points after injection using a Maestro in vivo imaging system (CRI, Woburn, Mass.; IRdye800 excitation=735 nm, emission=780 nm long pass).

### Example 10

Histologic Analysis: Bevacizumab (AVASTIN™)-treated LS174T tumor mice were injected with 1 nmol of Cy5.5-AVP. After 4 h blood circulation, the mice were injected with 200 μg FITC-labeled tomato lectin. The mice were sacrificed 10 min later, and the tumors collected and made into frozen tissue blocks. These tumor specimens were subsequently sectioned with a thickness of 10 μm. Fluorescence pictures were taken under a Zeiss microscope using FITC and Cy5.5 filter settings separately. Merged pictures were made using MetaMorph.

Histological detection of phage particles in tumor tissues with Cy5.5-labeled LLADTTHHRPWT-phage. Saline vehicle (A) or Bevacizumab (AVASTIN™)-treated (B) LS174T tumor mice were injected with 1 nmol of Cy5.5 conjugate. After 24 h circulation of the phage particles, FITC-conjugated tomato lectin was then injected for in vivo dual staining of tumor vasculature (100×). Cy5.5-AVP phage showed more tumor accumulation in Bevacizumab (AVASTIN™) treated than in saline control mice. Cy5.5 and FITC overlay pictures showed that AVP phages bind specifically to b Bevacizumab (AVASTIN™)-treated microvascular tumor endothelium cells. Note that the relatively weak Cy5.5 signal is likely due to the short circulation half-life of the phage particles.

### Example 11

Radiochemistry: Semi-preparative reversed-phase high performance liquid chromatography (RPHPLC) using a Vydac protein and peptide column (218TP510; 5 μm, 250×10 mm) was performed on a Dionex 680 chromatography system with a UVD 170U absorbance detector and model 105S single-channel radiation detector (Carroll & Ramsey Associates). The recorded data were processed using Chromeleon version 6.50 software. With a flow rate of 5 ml/min, the mobile phase was changed from 95% solvent A [0.1% trifluoroacetic acid (TFA) in water] and 5% B [0.1% TFA in acetonitrile] (0-2 min) to 35% solvent A and 65% solvent B at 32 min. Analytical HPLC had the same gradient system, except that the flow rate was 1 mL/min with a Vydac protein and peptide column (218TP510; 5 μm, 250×4.6 mm). The UV absorbance was monitored at 218 nm and the identification of the peptides was confirmed based on the UV spectrum acquired using a PDA detector. C18 Sep-Pak cartridges (Waters) were pretreated with ethanol and water before use.

FP-AVP was synthesized as follows: O—(N-Succinimidyl)-1,1,3,3-tetramethyluronium tetrafluoroborate (TSTU, 17.6 mg, 58.5 μmol) was added to a solution of 2-fluoropropionic acid (7.8 mg, 84.5 μmol) in 0.5 mL anhydrous acetonitrile. The pH of the solution was adjusted to 8.5-9.0 by N,N-Diisopropylethylamine (DIPEA). The reaction mixture was stirred at room temperature for 0.5 h and then AVP (AVASTIN™ (Bevacizumab) responsive peptide) (3 μmol) in DMF was added in one aliquot. After being stirred at room temperature for 2 h, the product FP-AVP was isolated by semi-preparative HPLC.

The collected fractions were combined and lyophilized to a white fluffy powder. FP-AVP was obtained in 82% yield with 22 min retention time on analytical HPLC. MALDI-TOF-MS was m/z 1522.1 for [MH]⁺ (C68H102FN20O19 calculated molecular weight 1522.7).

The labeling precursor 4-nitrophenyl 2-¹⁸F-fluoropropionate (¹⁸F-NFP) was synthesized as described by Liu et al., (2009) J. Med. Chem. 52: 425-432, incorporated herein by reference in its entirety). ¹⁸F-FP-AVP was synthesized as follows: AVP (1.0 pmol) and DIPEA (20 μL) were added to ¹⁸F-NFP in anhydrous dimethyl sulfoxide (DMSO, 200 μl). The reaction mixture was allowed to incubate at 60° C. for 20 min. After dilution with 2 ml of water 1.0% TFA, the mixture was injected into the semipreparative HPLC. The collected fractions containing ¹⁸F-FP-AVP were combined and rotary evaporated to remove acetonitrile and TFA. ¹⁸FFP-AVP was obtained in 15±4% yield (n=4). The activity was then reconstituted in normal saline and passed through a 0.22 μm Millipore filter into a sterile multidose vial for in vivo experiments.

### Example 12

Small Animal PET Imaging: A detailed procedure for positron emission tomography (PET) imaging has been reported earlier (Li et al., (2008) J. Nucl. Med. 49: 453-461). Briefly, PET scans were performed using a microPET R4 rodent model scanner (Siemens Medical Solutions). Mice were injected with about 100 μCi of ¹⁸F-FP-AVP or ¹⁸F-FDG via tail vein under isoflurane anesthesia and 3-5 min PET scans were performed at 1 h and 4 h post-injection (p.i.). The images were reconstructed by a two-dimensional ordered subsets expectation maximum (OSEM) algorithm with no attenuation or scatter correction. For each microPET scan, regions of interest (ROIs) were drawn over the tumor by using vendor software ASI Pro 5.2.4.0 on decay corrected whole-body coronal images. Assuming a tissue density of 1 g/ml, the ROIs were converted to MBq/g/min using a conversion factor, and then divided by the administered activity to obtain an imaging ROI-derived percent injected dose per gram (% ID/g).

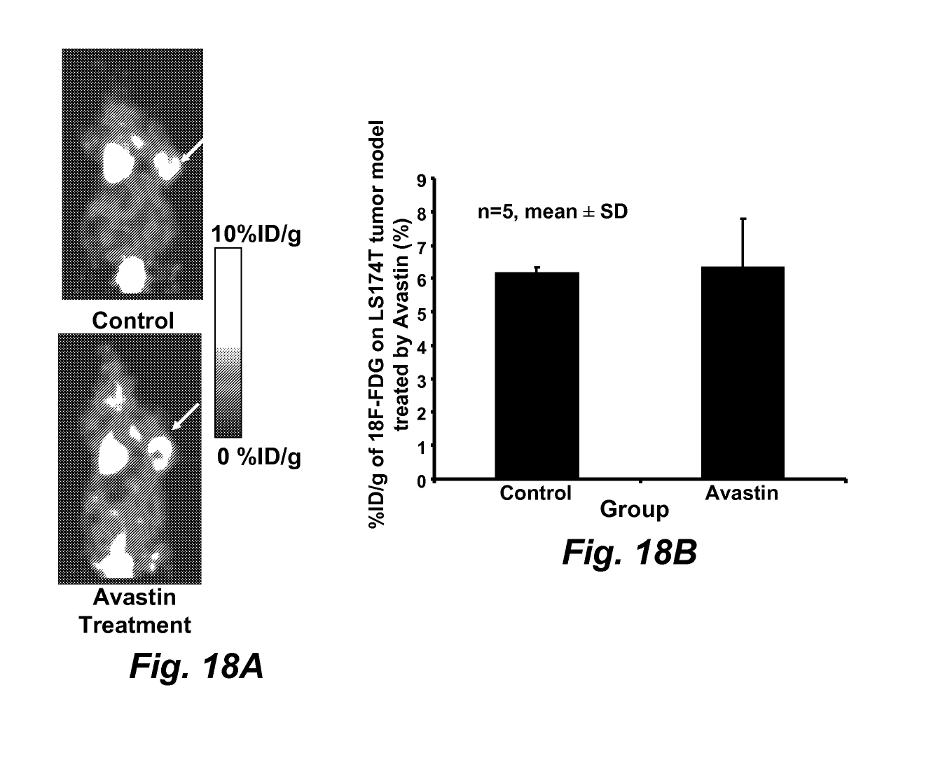

¹⁸F-FDG PET has been used routinely in both clinical and pre-clinical studies to measure glucose metabolism and, thereby, to evaluate stages of tumor progression and efficacy of therapeutic intervention (Gambhir et al., (2001) J. Nucl. Med. 42: 1S-93S). ¹⁸F-FDG small animal PET scans were carried out on an LS174T tumor model on the same day as ¹⁸F-FPP-AVP peptide administration (24 h after the 3rd dose of Bevacizumab (AVASTIN™), i.e. 5 days following the start of treatment). The heart had prominent uptake of ¹⁸F-FDG due to the constant beating, which has a high demand for glucose. The vehicle treated group and the Bevacizumab (AVASTIN™)-treated group had similar tumor uptake (6.16±0.15% ID/g versus 6.32±1.46 % ID/g, P=NS) (FIGS. 18A and 18B), indicating that the tumor cells remained viable during this early period of Bevacizumab (AVASTIN™) treatment.

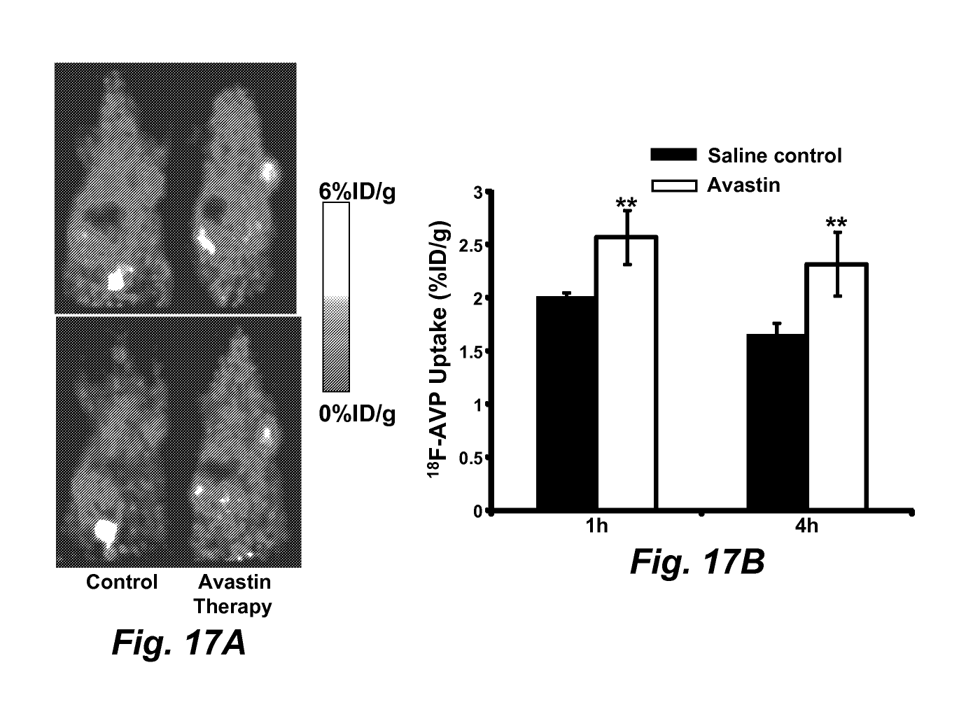

By contrast, the LS174T tumor uptake of ¹⁸F-FP-AVP increased from 2.02±0.04 % ID/g (control) to 2.63±0.25% ID/g (Bevacizumab (AVASTIN™)) at 1 h and from 1.59±0.11% ID/g (control) to 2.42±0.30% ID/g (Bevacizumab (AVASTIN™)) at the 4 h time point (P<0.05 for both time points) (FIGS. 17A and 17B). The increase in tumor uptake of ¹⁸F-FP-AVP is indicative of positive response to Bevacizumab (AVASTIN™) treatment.

### Example 13

Statistical Analyses: Statistical significance was determined by one-way ANOVA using an SPSS (10.0) statistics package. P value<0.05 was considered significant.

We claim:

1. A method for identifying an anti-angiogenic therapy-responsive peptide, comprising:
(a) providing a subject animal or human having a target tissue responsive to an anti-angiogenic therapeutic agent;
(b) administering an anti-angiogenic therapeutic agent to the subject animal or human;
(c) delivering to the subject animal or human a phage-displayed peptide library under conditions allowing at least one bacteriophage species from the phage-displayed peptide library to selectively bind to an anti-angiogenic therapy-responsive site of the target tissue;
(d) isolating the target tissue from the subject animal or human;
(e) isolating from the tumor a population of bacteriophages;
(f) amplifying said population of bacteriophages in a bacterial host and harvesting said amplified bacteriophages;
(g) repeating steps (b)-(f), thereby enriching the isolated phage population for peptide-bearing phage comprising an anti-angiogenic therapy-responsive peptide; and
(h) determining the nucleotide sequence encoding a phage-displayed peptide isolated by steps (a)-(g), thereby identifying a peptide positively responsive to an anti-angiogenic therapy.

2. The method of claim 1, wherein the tissue is a pathological tissue.

3. The method of claim 1, wherein the pathological tissue is a tumor.

4. The method of claim 1, wherein the anti-angiogenic therapeutic agent comprises an antibody or a fragment thereof.

5. The method of claim 1, wherein the anti-angiogenic therapeutic agent is bevacizmuab (AVASTIN™).

6. An isolated bacteriophage comprising an anti-angiogenic therapy-responsive peptide, wherein the isolated bacteriophage is characterized as selectively binding to a vascularized target tissue responsive to an anti-angiogenic therapeutic agent, or cells isolated therefrom.

7. The isolated bacteriophage of claim 6, wherein the vascularized target tissue is a pathological tissue.

8. The isolated bacteriophage of claim 6, wherein the pathological tissue is a tumor.

9. The isolated bacteriophage of claim 6, wherein the anti-angiogenic therapy-responsive peptide comprises an amino acid sequence selected from the group consisting of: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, wherein said peptide is characterized as having selective affinity for a vascularized target tissue responsive to an anti-angiogenic therapeutic agent, or cells isolated therefrom.

10. The isolated bacteriophage of claim 9, wherein the anti-angiogenic therapy-responsive peptide comprises the amino acid sequence selected from the group consisting of: LLADTTHHRPWT (SEQ ID NO.: 1), or a conservative variant thereof.

11. The isolated bacteriophage of claim 10, wherein the anti-angiogenic therapy-responsive peptide comprises the amino acid sequence selected from the group consisting of: LLADTTHHRPWT (SEQ ID NO.: 1).

12. The isolated bacteriophage of claim 6, further comprising a moiety or plurality of moieties attached to the bacteriophage and selected from the group consisting of: a therapeutic agent, a detectable label, and a combination thereof.

13. The isolated bacteriophage of claim 12, wherein the detectable label is selected from the group consisting of: a fluorescent label, a PET detectable label, an MRI-detectable label, and a radioactive label.

14. An isolated anti-angiogenic therapy-responsive peptide comprising an amino acid sequence selected from the group consisting of: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, wherein said peptide is characterized as having selective affinity for a site of a vascularized target tissue responsive to an anti-angiogenic therapeutic agent, or cells isolated therefrom.

15. The anti-angiogenic therapy-responsive peptide of claim 14, wherein the tissue is a pathological tissue.

16. The anti-angiogenic therapy-responsive peptide of claim 14, wherein the pathological tissue is a tumor.

17. The anti-angiogenic therapy-responsive peptide of claim 14 having the amino acid sequence SEQ ID NO.: 1.

18. The anti-angiogenic therapy-responsive peptide of claim 14, further comprising a moiety or plurality of moieties attached to the bacteriophage and selected from the group consisting of: a therapeutic agent, a detectable label, and a combination thereof.

19. The anti-angiogenic therapy-responsive peptide of claim 18, wherein the detectable label is selected from the group consisting of: a fluorescent label, a PET detectable label, an MRI-detectable label, and a radioactive label.

20. A composition selective for an anti-angiogenic therapy-responsive tissue in a subject animal or human comprising an anti-angiogenic therapy-responsive peptide linked to a moiety or plurality of moieties desired to be delivered to a tissue characterized as selectively binding anti-angiogenic therapy-responsive peptide, wherein said peptide is characterized as having selective affinity for a vascularized tissue responsive to an anti-angiogenic therapeutic agent, or cells isolated therefrom.

21. The composition of claim 20, wherein the vascularized tissue is a pathological tissue.

22. The composition of claim 20, wherein the pathological tissue is a tumor.

23. The composition of claim 20, further comprising a bacteriophage having anti-angiogenic therapy-responsive peptide expressed thereon, and wherein the moiety or plurality of moieties desired to be delivered to a tissue is optionally attached to the peptide or to the bacteriophage.

24. The composition of claim 20, wherein the anti-angiogenic therapy-responsive peptide is selected from the group consisting of the amino acid sequences: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof.

25. The composition of claim 24, wherein the anti-angiogenic therapy-responsive peptide has the amino acid sequence LLADTTHHRPWT (SEQ ID NO.: 1), or a conservative variant thereof.

26. The composition of claim 25, wherein the anti-angiogenic therapy-responsive peptide has the amino acid sequences: LLADTTHHRPWT (SEQ ID NO.: 1).

27. The composition of claim 20, wherein the moiety or plurality of moieties desired to be delivered to a vascularized target tissue and linked to the anti-angiogenic therapy-responsive peptide is selected from the group consisting of: a therapeutic agent, a detectable label, and a combination thereof.

28. The composition of claim 27, wherein the detectable label is selected from the group consisting of: a fluorescent label, a PET detectable label, an MRI-detectable label, and a radioactive label.

29. The composition of claim 20, further comprising a pharmaceutically acceptable carrier.

30. A method of imaging a tissue in an animal or human subject, comprising:
(a) delivering to a subject animal or human a pharmaceutically acceptable composition comprising an anti-angiogenic therapy-responsive peptide linked to a detectable label, whereby the anti-angiogenic therapy-responsive peptide is characterized as selectively concentrating at a site in a vascularized target tissue; and
(b) detecting the label in the subject animal or human, thereby identifying the location of an anti-angiogenic therapy-responsive vascularized target tissue in the host.

31. The method of claim 30, further comprising the steps of:
(i) delivering to the subject a therapeutic agent;
(ii) periodically imaging the vascularized target tissue in the subject; and
(iii) determining from the image size of the tissue whether the therapeutic agent is effective in reducing the size of the tumor in the subject.

32. The method of claim 30, wherein the target tissue is a pathological tissue.

33. The method of claim 30, wherein the pathological tissue is a tumor.

34. The method according to claim 30, wherein the anti-angiogenic therapy-responsive peptide is selected from the group consisting of the amino acid sequences: LLADTTHHRPWT (SEQ ID NO.: 1), SVSVGMKPSPRP (SEQ ID NO.: 2), LLADTTHHRPWP (SEQ ID NO.: 3), LLADATHHSPWP (SEQ ID NO.: 4), HSVSNIRPMFPS (SEQ ID NO.: 5), and SVSEGTHPSPRP (SEQ ID NO.: 6), or a conservative variant thereof, wherein said peptide is characterized as having selective affinity for a vascularized tumor responsive to an anti-angiogenic therapeutic agent, or cells isolated therefrom.

35. The method according to claim 30, wherein the anti-angiogenic therapy-responsive peptide has the amino acid sequence LLADTTHHRPWT (SEQ ID NO.: 1), or a conservative variant thereof.

36. The method according to claim 35, wherein the anti-angiogenic therapy-responsive peptide has the amino acid sequences: LLADTTHHRPWT (SEQ ID NO.: 1).

37. The method according to claim 30, wherein the anti-angiogenic therapy-responsive peptide is expressed by a bacteriophage and the detectable label is attached to the bacteriophage.

38. The method according to claim 37, wherein the detectable label is selected from the group consisting of: a fluorescent label, a PET detectable label, an MRI-detectable label, and a radioactive label.


SEQUENCE LISTING (7 sequences)

SEQ ID NO: 1 - protein, 12 aa, Artificial sequence
  Synthetic peptide
LLADTTHHRPWT

SEQ ID NO: 2 - protein, 12 aa, Artificial sequence
  Synthetic peptide
SVSVGMKPSPRP

SEQ ID NO: 3 - protein, 12 aa, Artificial sequence
  Synthetic peptide
LLADTTHHRPWP

SEQ ID NO: 4 - protein, 12 aa, Artificial sequence
  Synthetic peptide
LLADATHHSPWP

SEQ ID NO: 5 - protein, 12 aa, Artificial sequence
  Synthetic peptide
HSVSNIRPMFPS

SEQ ID NO: 6 - protein, 12 aa, Artificial sequence
  Synthetic peptide
SVSEGTHPSPRP

SEQ ID NO: 7 - protein, 12 aa, Artificial sequence
  Synthetic peptide
WTLRPTLHTDHA